(19) United States
(12) Patent Application Publication
(10) Pub. No.: US 2012/0156238 A1
(43) Pub. Date: Jun. 21, 2012

(54) HERPES VIRUS BACKBONE FOR VIRAL VACCINE AND VACCINE BASED THEREON

(76) Inventors: Saul J. Silverstein, Irvington, NY (US); Christos A. Panagiotidis, Ikismos Agias Anastasias (GR); Christos A. Kyratsous, New York, NY (US); Matthew S. Walters, New York, NY (US)

(21) Appl. No.: 13/387,692

(22) PCT Filed: Jul. 16, 2010
(86) PCT No.: PCT/US10/02010

§ 371 Date: Mar. 2, 2012

Related U.S. Application Data
(60) Provisional application No. 61/271,938, filed on Jul. 28, 2009.

(51) Int. Cl. A61K 39/12; C12N 7/01; A61P 31/22; A61K 39/245; A61P 37/04; C12N 15/38; A61K 39/25

(52) U.S. Cl. 424/199.1; 536/23.72; 435/235.1

(57) ABSTRACT

A recombinant deoxyribonucleic acid comprising a human herpes simplex virus (HSV) deoxyribonucleic acid (DNA) having a heterologous DNA integrated therein wherein the heterologous DNA encodes a polypeptide comprising a RING-finger domain; a recombinant virus comprising such, a viral vaccine and methods of immunization are provided.

This application claims the benefit of U.S. Provisional Application No. 61/271,938, filed Jul. 28, 2009, the entire content of which is hereby incorporated by reference herein.

The work disclosed herein was made with government support under grant no. AI-024021 from the National Institutes of Health. Accordingly, the U.S. Government has certain rights in this invention.

Throughout this application, certain publications are referenced. Full citations for these publications may be found immediately preceding the claims. The disclosures of these publications in their entireties are hereby incorporated by reference into this application in order to describe more fully the state-of-the art to which this invention relates.

### BACKGROUND

Viral vaccines were historically either inactivated viruses or live, attenuated viruses, but these types of vaccines have not been feasible to pursue for certain viruses such as herpes viruses or HIV. New types of vaccines including viral vectors, such as Herpes Simplex Virus-1 (HSV-1) are currently under investigation. In Herpes Simplex Virus expression of the HSV-1 protein ICP0 is required to allow efficient expression of genes from the viral genome and, more importantly, improve virus titer and thus vaccine yields. However, expression of ICP0 also interferes with innate immunity, a property which is not compatible with the ideal vaccine vector.

One of the functions of ICP0 is to dissociate cellular nuclear domain 10 (ND10) complexes and ubiquitinate the promyelocytic protein (PML) leading to its degradation. PML is one of the cell's gatekeepers that are responsible for induction of interferon.

Herein a recombinant virus employing the Herpes Simplex Virus genome as a backbone is disclosed, but with replacement of ICP0. The recombinant virus replicates to relatively high titers and is sensitive to interferon. Therefore, the vector only retains the properties of ICP0 that are required in vaccine development.

### SUMMARY OF THE INVENTION

A recombinant deoxyribonucleic acid comprising a human herpes simplex virus (HSV) deoxyribonucleic acid (DNA) having a heterologous DNA integrated therein wherein the heterologous DNA encodes a polypeptide comprising a RING-finger domain.

A recombinant human herpes simplex virus (HSV) comprising a heterologous DNA encoding a polypeptide comprising a RING-finger domain which heterologous DNA is (a) inserted into a non-essential region of the HSV genome, and (b) expressed in a host cell into which the recombinant HSV is introduced.

A vaccine comprising (1) a pharmaceutically acceptable carrier and (2) a recombinant virus which comprises a recombinant deoxyribonucleic acid comprising a human herpes simplex virus (HSV) genome having (a) a heterologous DNA encoding a polypeptide comprising a RING-finger domain integrated therein and (b) having one or more heterologous DNAs, each encoding a glycoprotein, integrated therein.

A method of immunizing a subject against a varicella zoster virus infection comprising administering to the subject an amount of the instant vaccine effective to elicit a immune response the varicella zoster virus in the subject and thereby effect immunization of the subject.

A method for preparing a composition useful for preventing infection by a virus, comprising combining the instant recombinant virus with a live vaccine stabilizer, so as to prepare the composition.

### BRIEF DESCRIPTION OF THE FIGURES

FIG. 1: Alignment of the amino acid sequences of ICP0 and ORF61p (SEQ ID NOs:1 (ICP0) and 2 (ORF61)). The amino acid sequences of ICP0 and ORF61p (NCBI protein database accession numbers NP_{—}044601.1 and NP_{—}040183.1 respectively) were aligned using the Gonnet Matrix, an Open Gap Penalty of 10.0 and an Extend Gap Penalty of 0.1. The Cys and His residues of the C3HC4 RING finger consensus sequence are marked d. The ND10 targeting domain (18) in ICP0's C-terminus of ICP0 are also marked. The NLS regions of ICP0 and ORF61p are underlined (52, 63). The USP7 interaction site is marked with a black box and the amino acids required for interaction are identified with arrows (19). The two regions that encode for E3 ubiquitin ligase activities (30) are marked also.

FIG. 2: Redistribution of PML and Sp100 in ICP0 or ORF61p expressing cells. MeWo cells grown on glass coverslips were mock treated (A and D) or transformed with plasmid constructs expressing ICP0 (B and E) or ORF61p (C and F). Forty-eight hours post transformation cells were fixed and the localization of viral proteins and PML (A-C) or Sp100 (D-F) were monitored by indirect immunofluorescence microscopy. The nuclei were stained with Hoechst. Images were captured with an 100× objective and analyzed by volume deconvolution.

FIG. 3: Abundance of PML and Sp100 in ICP0 or ORF61p expressing cells. MeWo cells were mock treated or infected with Adempty, AdICP0 or AdORF61 at an MOI of 5. Forty-eight hpi total cell lysates were analyzed by SDS-PAGE. The levels of PML, Sp100, ICP0, ORF61p and tubulin were analyzed by western blotting. The different species of PML and Sp100 are identified with asterisks.

FIG. 4: Targeting of ORF61p to ND10s does not lead to PML degradation. (A) Schematic diagram of ICP0, ORF61p and the ORF61p translational fusion to the C-terminus of ICP0. (B) MeWo cells were mock treated or transformed with constructs expressing ICP0, ORF61p or the ORF61p fusion protein. Forty-eight hpi total cell lysates were analyzed by SDS-PAGE. The levels of ICP0, ORF61p and tubulin were analyzed by western blotting. (C-E) MeWo cells grown on glass coverslips were mock treated (C) or transformed with plasmid constructs expressing the ORF61p fusion protein (D and E). After 48 hours cells were fixed and localization of ORF61p and PML were monitored by indirect immunofluorescence microscopy. Nuclei were stained with Hoechst. Images were captured with a 100× objective and analyzed by volume deconvolution.

FIG. 5: Localization of PML during HSV and VZV infection. MeWo cells grown on glass coverslips were infected with HSV at an MOI of 1 (A) or cell-free VZV at an MOI of approximately 0.01 (B-D). HSV infected cells were fixed at 6 hpi and VZV infected cells at 24 hpi and the presence of ICP0 (A), ORF61p (B), ORF62p (C), gE (D) and PML (A-D) were monitored by indirect immunofluorescence microscopy. Nuclei were stained with Hoechst. Images were captured with a 100× objective and analyzed by volume deconvolution.

FIG. 6: Localization of Sp100 during HSV and VZV infection. MeWo cells grown on glass coverslips were infected with HSV at an MOI of 1 (A) or cell-free VZV at an MOI of approximately 0.01 (B and C). HSV infected cells were fixed at 6 hpi and VZV infected cells at 24 hpi and the localization of ICP0 (A), ORF62p (B), and gE (C) and Sp100 (A-C) were monitored by indirect immunofluorescence microscopy. Nuclei were stained with Hoechst. Images were captured with a 100× objective and analyzed by volume deconvolution.

FIG. 7: Abundance of PML, Sp100 and Daxx during HSV and VZV infection. (A) MeWo cells were infected with HSV at an MOI of 5 or cell-free VZV at an MOI of approximately 0.01. At the indicated times post infection PML, Sp100, Daxx, ICP0, ORF61p and tubulin levels were monitored by western blotting. (B) MeWo cells were mock treated or incubated with media containing 1000 or 2000 U/ml interferon α. Forty-eight hours post treatment total cell lysates were monitored for PML, Sp100, Daxx, STAT1, phosphorylated STAT1, STAT2 and phosphorylated STAT2.

FIG. 8: Effect of PML, Sp100 and Daxx on VZV replication. (A) Total lysates from sicontrol, siPML, siSp100 and siDaxx cells were analyzed for PML, Sp100, Daxx and tubulin levels by western blotting. (B) sicontrol, siPML, siSp100 and siDaxx cells were infected with serial dilutions of VZV. Five days post infection monlayers were fixed and stained and plaques were counted and compared to the number formed in sicontrol cells. The error bars indicate standard deviation from five independent experiments, each performed in duplicate. (C) sicontrol, siPML, siSp100 and siDaxx cells were infected with cell-free VZV at an MOI of approximately 0.01. At the indicated times post infection lysates were analyzed by western blotting for ORF29p, ORF63p and tubulin. Band intensities were quantified using ImageJ and normalized to tubulin. (D) sicontrol, siPML, siSp100 and siDaxx cells were infected with cell-free VZV at an MOI of approximately 0.01. At the indicated times post infection cells were harvested and titrated to determine the number of infectious centers on fresh MeWo monolayers. Four days post infection monolayers were fixed and stained and plaques were counted to calculate the titer of infectious centers. Panel D is a representative experiment showing a growth curve in each of the cell lines. Each data point represents the average of two samples. Panel E displays the averages of the time points from four independent experiments and error bars represent standard deviation.

FIG. 9: Morphology of infected foci on sicontrol, siPML, siSp100 and siDaxx cells. sicontrol, siPML, siSp100 and siDaxx cells were infected with cell-free VZV at an MOI of approximately 0.01. At 2 and 3 dpi the cells were fixed and the localization of ORF63p and gE were monitored by immunofluorescence microscopy. Nuclei were stained with Hoechst. Images were captured with a 10× objective.

FIG. 10: Construction of a Herpes Simplex Virus expressing Varicella Zoster Virus ORF61p. (A) Schematic diagram of the ICP0 locus. (B) pCPC-061 and Hirt DNA prepared from dl1403 or HSV-ORF61 infected cells were amplified using the primer sets specified in Materials and Methods of results II hereinbelow. (C) MeWo cells were either mock treated or infected with wild-type HSV-1, dl1403 or HSV-ORF61 at an MOI of 5. At 8 hpi cells were harvested and western blotting using the antibodies described in Materials and Methods monitored the abundance of ICP0, ICP4, ORF61p and tubulin.

FIG. 11: Growth analysis of Herpes Simplex Virus expressing Varicella Zoster Virus ORF61p. (A) Vero or L7 cells were infected with serial dilutions of wild-type HSV-1, dl1403 or HSV-ORF61. Three days post infection monolayers were fixed and stained, and plaques were counted. Relative plaquing efficiency is: ×100. The error bars indicate standard deviation from four independent experiments, each performed in duplicate. (B) MeWo cells were infected with wild-type HSV-1, dl1403 or HSV-ORF61 at an MOI of 0.1. At 2, 12, 24 and 48 hpi infected cells were harvested, subjected to three rounds of freeze-thaw and yields were calculated after titration on L7 cells.

FIG. 12. Time course of expression of virus-specified proteins. MeWo cells were infected with wild-type (HSV-1), ICP0-(dl1403) and HSV-ORF61 at a moi of 0.2. Infected cells were harvested at the indicated times and examined for the synthesis and abundance of ICP4, ICP27, ICP0 and ORF61p by western blot. All lanes were stained with anti-tubulin antibody to demonstrate that equivalent amounts of cell protein were loaded in each lane.

FIG. 13: The fate and requirement of PML and Sp100 during infection with a Herpes Simplex Virus expressing Varicella Zoster Virus ORF61p. (A) MeWo cells were either mock treated or infected with wild-type HSV-1, dl1403 or HSV-ORF61 at an MOI of 10. At 2 and 4 hpi cells were harvested and western blotting was used to monitor the abundance of PML, Sp100, ICP0, ORF61p and tubulin. (B) Empty, siPML and siSp100 cells were infected with serial dilutions of wild-type HSV-1, dl1403 or HSV-ORF61. Three days post infection monolayers were fixed and stained, and plaques were counted and compared to the number formed in empty cells. The error bars represent standard deviation from three independent experiments, each performed in duplicate.

FIG. 14: Sensitivity of a Herpes Simplex Virus expressing Varicella Zoster Virus ORF61p to interferon α. MeWo, Vero or U2OS cells that were mock treated or treated overnight with interferon α were infected with serial dilutions of wild-type HSV-1, dl1403 or HSV-ORF61. Three days post infection monolayers were fixed and stained, and plaques were counted. The relative plaquing efficiency of each virus on each cell line was calculated as the titer in mock treated cells/the titer in interferon α treated cells×100.

FIG. 15: Schematic representation of the VZV genome. ORFs encoding glycoproteins are identified.

### DETAILED DESCRIPTION OF THE INVENTION

Non-limiting examples of HSV include Glasgow strain 7; Human herpesvirus 1 (HSV-1) HF VR-260™; Human herpesvirus 1 (HSV-1) MacIntyre VR-539™; Human herpesvirus 1 (HSV-1) KOS VR-1493™; Human herpesvirus 2 (HSV-2) G VR-734™; Human herpesvirus 2 (HSV-2) MS VR-540™; and Human herpesvirus, recombinant GHSV-UL46 VR-1544™ as deposited with the ATCC at Manassas, Va. 20108, USA. Non-limiting examples of VZV include Jones strain; Oka strain of VZV, VR-795 as deposited with the ATCC at Manassas, Va. 20108, USA.

“Recombinant” as used herein, in reference to a nucleic acid or virus, means a molecular or viral entity having two separate sources of origin combined into a single form of nucleic acid or virus, respectively. For example, a recombinant deoxyribonucleic acid could be made from a herpes simplex virus genome and a gene from a varicella zoster virus both combined into a single DNA molecule. For example, a recombinant virus is a virus having therein a nucleic acid which contains a foreign or heterologous coding sequence/gene. In an embodiment, the two different courses are different species.

“Heterologous” as used herein, in reference to e.g. a heterologous deoxyribonucleic acid, means derived from a different organism (or having a sequence identical thereto) than the DNA of the organism to which the DNA is described as heterologous relative to. In a non-limiting example, a DNA derived from a varicella zoster virus inserted into a DNA derived from a herpes simplex virus would be heterologous DNA relative to the DNA derived from the herpes simplex virus.

“RING finger domain” shall mean a zinc finger polypeptide which containing a Cys3HisCys4 amino acid motif which binds two zinc cations. The acronym RING stands for Really Interesting New Gene.

“Integrated” as used herein with regard to a heterologous gene or coding sequence being integrated into a viral DNA shall mean the functional inclusion of the heterologous gene or coding sequence into the DNA such that the gene or coding sequence is expressed when the viral DNA is expressed by a host cell infected by the virus.

A “non-essential” gene or region of a viral DNA is a region of DNA, known to those skilled in the art, the insertion of a DNA sequence into which does not prevent the virus's ability to infect a host cell and replicate therein.

Varicella zoster virus glycoproteins and nucleic acid sequences encoding them are well known in the art, e.g. glycoproteins H, B and E (gH, gB and gE, respectively), for example see Maresova et al. 2005, J. Virol. 79 (2): 997-1007, which is hereby incorporated by reference in its entirety.

Varicella zoster virus neutralizing epitopes are well known in the art, for example see Akahori et al., Journal of Virology, February 2009, p. 2020-2024, Vol. 83, No. 4; and Forghani et al. J Clin Microbiol. 1990 November; 28(11): 2500-2506, each of which are hereby incorporated by reference in their entirety.

Infected Cell Polypeptide 0 (ICP0) is a protein, encoded by the DNA of herpes viruses (see NCBI protein database accession number NP_{—}044601.1, hereby incorporated by reference in its entirety). ORF61 is open reading frame 61 protein (varicella zoster virus), (see NCBI protein database accession number NP_{—}040183.1, hereby incorporated by reference in its entirety).

Immunization as used herein is the presentation of viral antigen(s) (in a vaccine for example) to the immune system of a subject, for example a human, which provokes an immune response and evokes protective humoral and/or cellular immunity in subsequent exposure to the viral antigen(s).

Compositions for viral vaccines are known to those in the art and are described in U.S. Pat. No. 6,616,931, Burke, et al. Sep. 9, 2003; U.S. Pat. No. 6,258,362, Loudon et al., Jul. 10, 2001; and U.S. Pat. No. 6,787,351, Chen, et al. Sep. 7, 2004, each of which are hereby incorporated by reference in their entirety. The viral vector vaccine disclosed herein can be administered in a pharmaceutically acceptable solution, such as, but not limited to, sterile saline or sterile buffered saline and can be administered with or without adjuvants for vaccines known in the art. “Administering” a vaccine can be effected or performed using any of the various methods and delivery systems known to those skilled in the art. The viral vaccines can be administered by methods known in the art including, but not limited to, by injection, topically, and to mucous membranes/nasal membranes.

As used herein, a “pharmaceutical carrier” is a pharmaceutically acceptable solvent, suspending agent or vehicle, for delivering the instant viral vectors to the animal or human. The carrier may be liquid, aerosol, gel or solid and is selected with the planned manner of administration in mind. In an embodiment, the pharmaceutical carrier is a sterile pharmaceutically acceptable solvent.

Methods of virus vaccine production and storage are known in the art and are also described in WO/2006/012092, which is hereby incorporated by reference in its entirety. As described therein, stabilizers often used for live vaccines of viruses such of measles, rubella and mumps generally include one or more saccharides, amino acids, sugar alcohols, gelatin and gelatin derivatives, to stabilize the virus and, in many cases keep the virus from denaturing during a concentration step. The recombinant virus described herein may be formulated into a vaccine using a stabilizer or other additive that includes native or recombinant serum albumin for this purpose. U.S. Pat. Nos. 6,210,683; 5,728,386, 6,051,238, 6,039,958 and 6,258,362 also contain details for stabilizers and methods for more gentle treatment of live virus vaccines. Each of these disclosures, and particularly those portions that describe stabilizer compositions and stabilizing methods are specifically incorporated by reference in their entireties. After preparation with a stabilizer, the vaccine may be, for example, stored as a lyophilized vaccine, a lyophilized mixed vaccine, a liquid vaccine or a liquid mixed vaccine. Methods for forming these are known. Typically, a lyophilized vaccine is prepared by lyophilizing the vaccine in a vial or an ampule having a volume of about 3 to 30 ml, tightly sealing and storing at a temperature of 5 degrees Centigrade or less. The stored preparation vaccine typically is used according to instructions attached thereto, as a product insert or a notice on the vial or other container. In many cases, a lyophilized vaccine is re-constituted by addition of sterile distilled water before use, and the resultant solution is inoculated by hypodermic injection in an amount, for example, of 0.5 ml per dose. The vaccine can also be provided orally or nasally.

A recombinant deoxyribonucleic acid comprising a human herpes simplex virus (HSV) deoxyribonucleic acid (DNA) having a heterologous DNA integrated therein wherein the heterologous DNA encodes a polypeptide comprising a RING-finger domain.

In an embodiment the HSV DNA is genomic DNA and the heterologous DNA is integrated into the HSV DNA in place of a portion of genomic HSV DNA which encodes a HSV Infected Cell Polypeptide 0 (ICP0). In an embodiment the heterologous DNA is inserted into the genomic HSV DNA between a HSV ICPO 5′ untranslated region (UTR) and a HSV ICPO 3′ untranslated region (UTR). In an embodiment the inserted heterologous DNA is under the control of the ICP0 promoter and 3′UTR. In an embodiment the polypeptide comprising the RING-finger domain has the amino acid sequence of varicella zoster virus ORF61 protein. In an embodiment the heterologous polypeptide comprising the RING-finger domain has the amino acid sequence of a equine herpes virus, bovine herpes virus, or pseudorabies virus protein. In an embodiment the HSV DNA is a HSV genome.

In an embodiment the recombinant deoxyribonucleic acid further comprises a heterologous DNA encoding a glycoprotein. In an embodiment the recombinant deoxyribonucleic acid further comprises up to eight heterologous DNAs each encoding a different glycoprotein. In an embodiment the glycoprotein has the amino acid sequence of a varicella zoster virus glycoprotein. In an embodiment the glycoprotein comprises a varicella zoster virus neutralizing epitope.

In an embodiment no polypeptide encoded by the recombinant deoxyribonucleic acid degrades mammalian promyelocytic leukemia protein (PML).

In an embodiment the heterologous DNA encoding the polypeptide is integrated such that the polypeptide is expressed when the recombinant deoxyribonucleic acid is integrated into a genome of a suitable host cell. In an embodiment the heterologous DNA encoding the glycoprotein is inserted into a non-essential gene or region of the HSV DNA.

A recombinant human herpes simplex virus (HSV) comprising a heterologous DNA encoding a polypeptide comprising a RING-finger domain which heterologous DNA is (a) inserted into a non-essential region of the HSV genome, and (b) expressed in a host cell into which the recombinant HSV is introduced.

In an embodiment the heterologous DNA is integrated into the HSV genome in place of a portion of the HSV genome which encodes a HSV Infected Cell Polypeptide 0 (ICP0). In an embodiment the heterologous DNA is inserted into the HSV genome between a HSV ICPO 5′ untranslated region (UTR) and a HSV ICPO 3′ untranslated region (UTR). In an embodiment the polypeptide comprising the RING-finger domain has the amino acid sequence of varicella zoster virus ORF61 protein. In an embodiment the polypeptide comprising the RING-finger domain has the amino acid sequence of a equine herpes virus, bovine herpes virus, or pseudorabies virus protein. In an embodiment the recombinant HSV further comprises a heterologous DNA encoding a glycoprotein and inserted into a non-essential region of the HSV genome. In an embodiment amino acid sequence of a varicella zoster virus glycoprotein. In an embodiment the glycoprotein comprises a varicella-zoster virus neutralizing epitope. In an embodiment the heterologous DNA encoding the glycoprotein is inserted into a non-essential gene or region of the HSV genome. In an embodiment none of the polypeptides encoded by the recombinant HSV genome degrade PML. In an embodiment the recombinant HSV comprises up to eight different heterologous DNAs, each encoding a different varicella zoster virus glycoprotein. In an embodiment the recombinant virus is attenuated by those methods known in the art.

A vaccine comprising (1) a pharmaceutically acceptable carrier and (2) a recombinant virus which comprises a recombinant deoxyribonucleic acid comprising a human herpes simplex virus (HSV) genome having (a) a heterologous DNA encoding a polypeptide comprising a RING-finger domain integrated therein and (b) having one or more heterologous DNAs, each encoding a glycoprotein, integrated therein.

In an embodiment the heterologous DNA is integrated into the HSV genome in place of a portion of HSV DNA encoding a HSV Infected Cell Polypeptide 0 (ICP0). In an embodiment the heterologous DNA is inserted into the HSV genome between a HSV ICPO 5′ untranslated region (UTR) and a HSV ICPO 3′ untranslated region (UTR). In an embodiment the heterologous polypeptide comprising a RING-finger domain has the amino acid sequence of varicella zoster virus ORF61 protein. In an embodiment the heterologous polypeptide comprising a RING-finger domain has the amino acid sequence of EHV, BHV, or pseudorabies virus protein. In an embodiment the glycoprotein has the amino acid sequence of a varicella zoster virus glycoprotein. In an embodiment the glycoprotein comprises a varicella-zoster virus neutralizing epitope. In an embodiment the heterologous DNAs encoding the glycoproteins and the a heterologous DNA encoding the polypeptide comprising the RING-finger domain are each inserted into non-essential genes or regions of the HSV genome. In an embodiment the recombinant virus is attenuated by those methods known in the art.

A method of immunizing a subject against a varicella zoster virus infection comprising administering to the subject an amount of the instant vaccines effective to elicit a immune response the varicella zoster virus in the subject and thereby effect immunization of the subject.

A method for preparing a composition useful for preventing infection by a virus, comprising combining one of the instant recombinant viruses with a live vaccine stabilizer, so as to prepare the composition.

In an embodiment of the methods described herein the subject is mammalian. In an embodiment the subject is human. In an embodiment of the methods the host cell is mammalian or derived from a mammal. In an embodiment the host cell is obtained from a human.

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Studies disclosed below show that the corresponding ortholog of HSV ICP0 in Varicella Zoster Virus (VZV), ORF61, is unable to degrade PML and thus does not interfere with the host innate immunity. Herein, construction of a recombinant HSV is disclosed wherein the coding sequence for the α gene ICP0 is replaced with a sequence encoding ORF61p.

### Results I

Nuclear domains 10 (ND10s), also known as PML nuclear bodies and PML oncogenic domains, are dynamic macromolecular inclusions of cellular proteins that form within the interchromosomal space in the nucleus (2, 65). The size and frequency of these bodies range from 0.2 to 1 um and from 2 to 30 per cell, respectively, depending on cell type and stage of the cell cycle (2, 17, 62). Cellular proteins that accumulate at these sites are divided into two groups: proteins that are permanent components, such as PML (promyelocytic leukemia protein), Sp100 (speckled protein of 100 kDa), Daxx, SUMO-1 and the Bloom syndrome helicase BLM, and proteins that only associate with ND10s under specific conditions (e.g. DNA repair machinery) or overexpression (e.g. BRCA1) (70).

DNA virus genomes associate with ND10 components at the initial stages of their replication cycles. Newly formed transcription and replication sites localize close to proteins that normally reside within ND10s (61). The first suggestion that virus replication affected ND10s was the demonstration that PML staining disappeared after Herpes Simplex Virus (HSV) infection (46). Subsequently, parental genomes of herpesviruses, adenoviruses, simian virus 40 (SV40) and papillomaviruses were shown to be associated with ND10s (10, 14, 32, 33, 35, 47).

The antagonistic relationship between HSV and components of ND10s has been extensively studied. Expression of ICP0, a viral protein, is required and sufficient for destruction of the nuclear structure (18, 45, 46). ICP0 is a C₃HC₄ RING finger containing, nuclear phosphoprotein with an apparent molecular mass of 110 kDa (56), that behaves as a promiscuous activator of both viral and cellular genes (12, 23, 59). Virus mutants lacking the ICP0 gene have an increased particle to plaque forming unit (pfu) ratio, substantially lower yield and decreased levels of α gene expression (13, 64). ICP0 also functions as an E3 ubiquitin ligase to target a growing list of host proteins for proteasomal degradation, including components of ND10 bodies, such as the SUMO-1 modified forms of PML and Sp100 (8, 15, 18, 26, 41, 53).

HSV mutants that fail to express ICP0 are defective in their ability to modify and degrade ND10 components (46). Depletion of PML and Sp100 accelerated virus gene expression and increased plaquing efficiency of HSV ICP0 defective viruses, but had no effect on wild-type virus. These data show that PML and Sp100 are components of an intrinsic anti-HSV defense mechanism that is counteracted by ICP0's E3 ligase activity to ensure efficient virus replication and growth (21, 22).

Varicella-Zoster Virus (VZV) is a common human pathogen that is classified together with HSV as an alphaherpesvirus. VZV encodes an ICP0 ortholog (ORF61p) (49, 57) that, similar to ICP0, transcriptionally activates viral promoters and enhances infectivity of viral DNA (49, 50). Importantly, ORF61p contains a RING finger domain, homologous to the one that is essential for ICP0's transactivation and ND10 dissociation and degradation activities.

It has previously been shown that expression of ICP0 by HSV is required to overcome depletion of BAG3, a host co-chaperone protein that stimulates virus gene expression and protein accumulation (38). Although ORF61p is considered functionally similar to ICP0 (49), VZV is affected by depletion of BAG3 (37), suggesting that ICP0 and ORF61p have evolved separately to provide different functions for virus replication.

This report demonstrates that ORF61p and other VZV encoded proteins do not degrade ND10 components in the same manner as does ICP0 in HSV infected cells. The role of PML, Sp100 and Daxx during VZV infection were also studied, and this report highlights key differences between the two related alphaherpesviruses.

Materials and Methods: Mammalian cells. Human melanoma (MeWo), siBAG3 (37), siPML (38) and 293A cells were maintained as previously described (37). To generate stable cell lines expressing siRNAs targeting Sp100 and Daxx mRNAs, MeWo cells were infected with retroviruses and selected in growth medium containing 200 ug/ml and then 500 ug/ml hygromycin.

DNA transformation. DNAs were transformed into the appropriate cell lines using Fugene HD [Roche, Indianapolis, Ind.].

Drug treatment. Interferon α was purchased from PBL Biomedical [Piscataway, N.J.].

Viruses. [i] VZV. Jones, a wild-type clinical isolate, was propagated and titrated as described (24). Cell-free virus was prepared as described (36, 58). [ii] Retroviruses. Retroviruses were constructed by transient co-transformation of 293T cells with the proviral vectors pCK-Super.retro.hygro (38), pCK-siSp100 or pCK-siDaxx and pgag-polgpt (44) and pHCMV-G (75). [ii] Adenoviruses. Adenoviruses Adempty, AdICP0 and AdORF61 were previously described (74, 76).

Virus growth assays. [i] Plaque assays. MeWo, siPML, siSp100 or siDaxx cells were infected with 10-fold serial dilutions of virus stocks and infected cells were fixed, stained and plaques counted. [ii] Growth curves. The titer of cell associated VZV after infection of MeWo, siPML, siSp100 or siDaxx cells was determined by mixing infected cells with uninfected MeWo cells and counting the resulting plaques after fixing and staining.

Plasmids construction. [i] siRNA plasmids. The previously described siRNA oligos targeting Sp100 and Daxx mRNA (22, 68) were modified for cloning into pCK-super.retro.hygro (38). To generate pCK-siSp100 and pCK-siDaxx the annealed oligo pairs siSp100_upper: 5′-GATCCCCGTGAGCCTGTGATCAATAATTCAAGAGATTATTGATCACAGGCTCACTT TTTA-3′ and siSp100_lower: 5′-AGCTTAAAAAGTGAGCCTGTGATCAATAATCTCTTGAATTATTGATCACAGGCTCA CGGG or siDaxx_upper: 5′-GATCCCCGGAGTTGGATCTCTCAGAATTCAAGAGATTCTGAGAGATCCAACTCCTT TTTA-3′ siDaxx_lower: 5′-AGCTTAAAAAGGAGTTGGATCTCTCAGAATCTCTTGAATTCTGAGAGATCCAACTC CGGG-3′ were ligated into BglII/HindIII cleaved pCK-super.retro.hygro (38).

All primers were obtained from by Operon Biotechnologies [Huntsville, Ala.] and all vector inserts were verified by DNA sequencing.

Antibodies. Rabbit polyclonal antibodies against amino acids [aa] 1086 to 1201 of ORF29p and aa 1-265 of ORF63p were described (43).

Polyclonal antibodies to ICP0 have been described (40). Monoclonal ICP0 and ORF62p antibodies were purchased from the Rumbaugh-Goodwin Institute [Plantation, Fla.].

Polyclonal antibodies against a GST-fusion protein containing amino acids 136-248 of ORF61p were raised in rabbits and purified by affinity chromatography as described before (37). Monoclonal antibodies to PML, GAPDH and tubulin and polyclonal antibodies to Daxx were obtained from Santa Cruz Biotechnology [Santa Cruz, Calif.]. Polyclonal antibodies against PML and Sp100 were purchased from Chemicon [Temecula, Calif.]. Antibodies to STAT1, STAT2 and the phosphorylated STAT1 were obtained from Abcam [Cambridge, Mass.]. Antibodies against phosphorylated STAT2 were purchased from Santa Cruz Biotechnology.

Alexa Fluor 488-conjugated anti-mouse and Alexa Fluor 546-conjugated anti-rabbit antibodies were obtained from Molecular Probes [Carlsbad, Calif.]. Goat anti-rabbit and anti-mouse antibodies conjugated to horseradish peroxidase for immunoblotting were obtained from KPL [Gaitherburg, Md.].

Indirect immunofluorescence microscopy. Cells on glass coverslips were fixed and stained with antibody and Hoechst as previously described (37). All samples were visualized with a Zeiss Axiovert 200M inverted microscope [Carl Zeiss Microimaging Inc, Thornwood, N.Y.] and images were acquired with a Hamamatsu C4742-80-12AG Digital CCD Camera [Hamamatsu Photonics, Hamamatsu-City, Japan] using Openlab 5 software [Improvision, Lexington, Mass.]. Images were deconvolved using Openlab 5 and assembled in Photoshop CS3 [Adobe Systems, San Jose, Calif.].

SDS-PAGE and western blotting. Infected or biochemically transformed cells were washed twice with cold PBS, lysed in 1.5×SDS sample buffer [75 mM TrisHCl pH 6.8, 150 mM DTT, 3% SDS, 0.15% bromophenol blue, 15% glycerol], boiled and proteins analyzed by SDS-PAGE (39). Proteins were transferred to nitrocellulose membranes before western blotting. After blocking membranes in 5% non-fat milk in PBST, immobilized proteins were reacted with the appropriate antibodies in 1% nonfat milk in PBST. Membranes were washed three times for 5 min each with PBST, incubated with an anti-rabbit or anti-mouse antibody conjugated to horseradish peroxidase, then washed again three times for 5 min with PBST and twice with PBS. Antibodies were visualized by addition of LumiGLO substrate [KPL] and exposure to X-ray film.

When the antibodies against the STAT proteins were used, blocking and antibody incubations were performed in PBST supplemented with 50 mM NaF and 1 mM Na₃VO₄ and 3% BSA.

Sequence alignment. The amino acid sequences of ORF61p (NP_{—}040183.1) and ICP0 (NP_{—}044601.1) were aligned with MacVector ver 10.0 [MacVector Inc, Cary, N.C.] using the Gonnet Matrix, an Open Gap Penalty of 10.0 and an Extend Gap Penalty of 0.1.

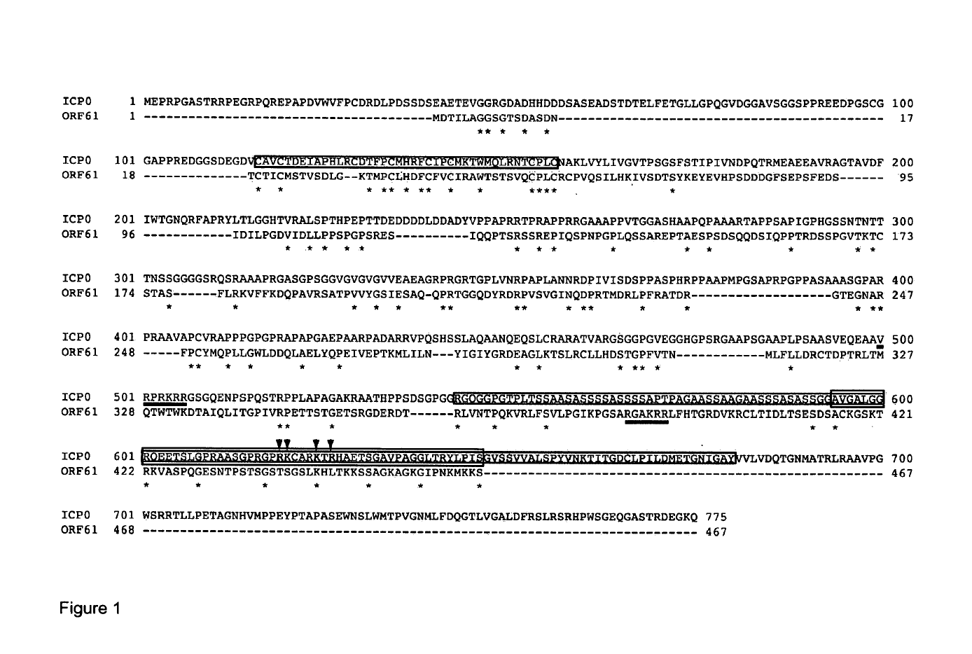

Sequence similarities between ICP0 and ORF61p. Previous reports suggested that the alphaherpesvirus orthologs, ICP0 and ORF61p, share several biological features, including their abilities to function as activators of gene expression and enhancers of viral DNA infectivity (49, 50, 57). However, it has also been reported that unlike ICP0, ORF61p did not substitute for loss of BAG3 during virus infection, suggesting that these proteins do not serve identical functions in their native context (38). Multiple functions of ICP0 have been mapped to specific domains (16). This led to the hypothesis that alignment of the amino acid sequences between these proteins might provide insight into their differences. The amino acid sequences of ICP0 and ORF61p were fetched from the NCBI protein database (accession numbers NP_{—}044601.1 and NP_{—}040183.1, respectively) and aligned (FIG. 1). Despite some conservation of their nucleotide sequence (9), there was found to be little overall amino acid conservation between them (10% similarity at the amino acid level). However, both contain a C₃HC₄ RING finger [C-X2-C-X(9-39)-C-X(1-3)-H-X(2-3)-C-X2-C-X(4-48)-C-X2-C] (3) close to their N-termini. A striking difference between these two proteins is the absence of the ICP0 C-terminus (marked in blue) in the sequence of ORF61p. Importantly, this region is required for targeting ICP0 to ND10 bodies and subsequently degrading their components (16, 18, 45). Therefore, an investigation of the ability of ORF61p to mediate degradation of PML and Sp100 was performed.

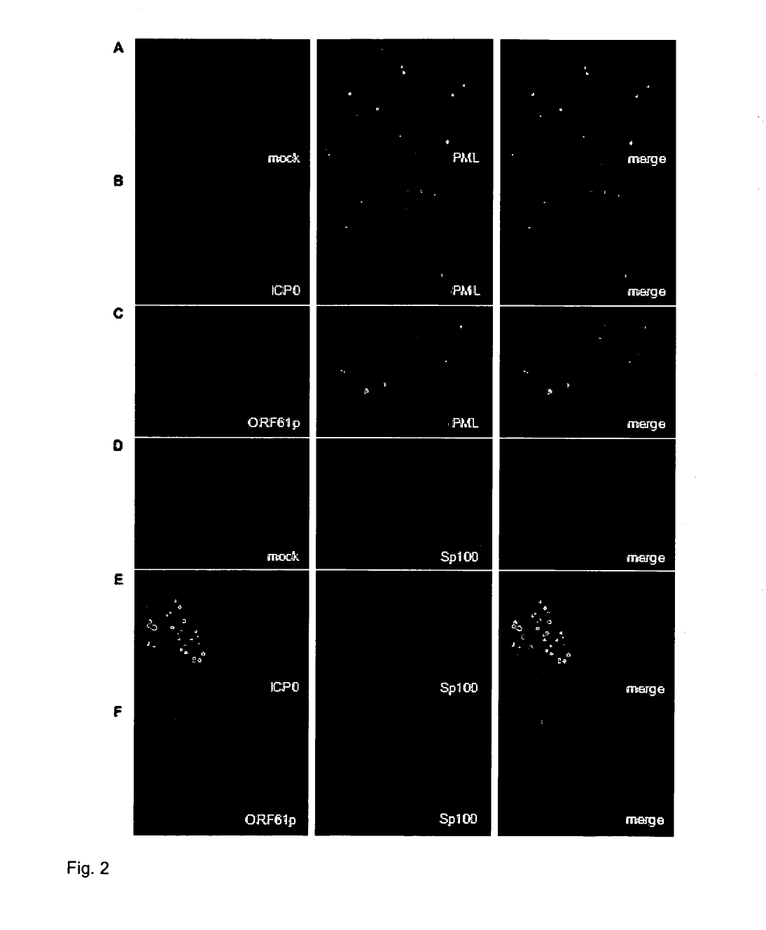

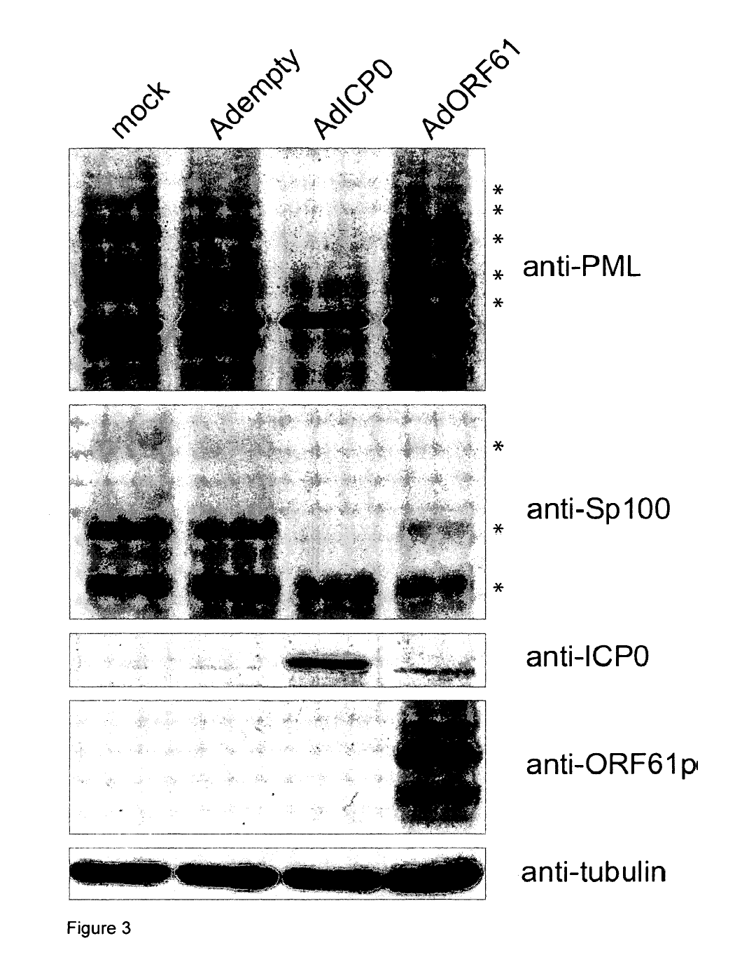

ORF61p is unable to efficiently degrade PML and Sp100. The effects of ORF61p and ICP0 on localization of PML and Sp100 in MeWo cells transformed with plasmids encoding these two orthologs were monitored using immunofluorescence microscopy. In mock treated cells both PML and Sp100 appeared as punctate nuclear bodies (FIGS. 2A and 2D) (2). As previously reported, expression of ICP0 resulted in disappearance of both cell proteins (FIGS. 2B and 2E) (46). However, cells expressing ORF61p displayed a different phenotype. Although PML containing bodies were more dispersed and smaller when compared to mock treated cells, the protein was still detected, mostly in punctate intracellular structures (FIG. 2C). The effect of ORF61p on Sp100 seemed more similar to that of ICP0, as staining for Sp100 nuclear bodies disappeared from most cells expressing ORF61p (FIG. 2F). To directly assay the effect of virus products on PML and Sp100 abundance, MeWo cells were mock treated or infected with replication deficient adenoviruses expressing either no herpesvirus proteins (Adempty), ICP0 (AdICP0) or ORF61p (AdORF61), and viral and cellular protein levels were monitored by western blotting (FIG. 3). Using a multiplicity of infection (MOI) of 5 ensured that every cell was infected and expressed the protein of interest. In mock infected cells or cells infected with Adempty, PML appeared as a series of bands with different molecular masses. These represented alternatively spliced and post-translationally modified forms of PML (54). Three predominant forms of Sp100 (Sp100A, Sp100A-SUMO and Sp100-HMG) were detected as previously described (27, 66). ICP0 expression resulted in complete disappearance of multiple PML isoforms and preferential loss of the slower migrating species of Sp100 (FIG. 3) (8). In contrast, expression of ORF61p had no effect on abundance or species of PML detected by this assay. ORF61p consistently reduced Sp100 levels, although not as efficiently as did ICP0 (FIG. 3). Immunofluorescence analysis of cells infected in parallel verified that all cells were infected and expressed ICP0 or ORF61p (data not shown). PML in cells infected with Adempty was reorganized as elongated tracks, presumably in response to expression of adenovirus E4 ORF3 (6). Expression of ICP0 by AdICP0 led to loss of PML, whereas PML staining in AdORF61 infected cells was identical to the Adempty sample (data not shown). This observation further differentiates ICP0 from ORF61p.

Taken together, the immunofluorescence data and the western blotting analysis demonstrate that although ORF61p alters integrity and the staining pattern of ND10 bodies, its expression does not result in the disappearance of PML. However, ORF61p expression but does affect Sp100 levels.

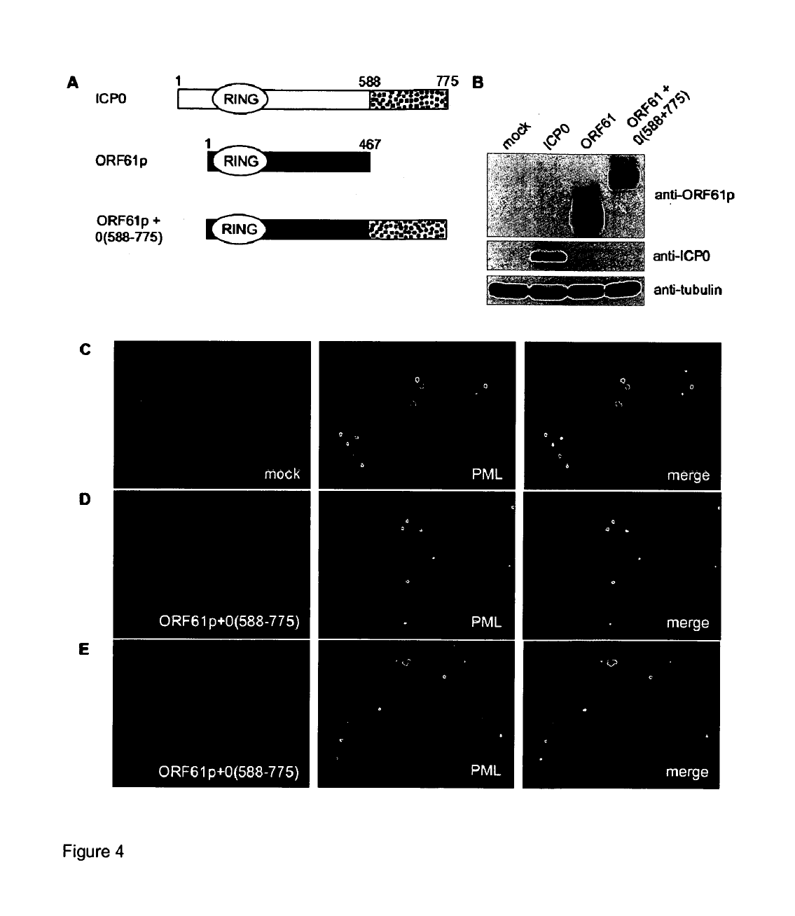

Targeting of ORF61p to ND10s does not cause PML degradation. As mentioned above, the C-terminus of ICP0, which is required for targeting to ND10s and efficient degradation of PML (16, 18, 45), is absent from ORF61p (FIG. 1). Addition of this domain to β-galactosidase caused its partial co-localization with PML (18). To test whether ORF61p's failure to decrease PML levels was a consequence of its inability to target ND10s because it lacked this domain, a translational fusion of ORF61p with the C-terminal 188 amino acids of ICP0 was created (FIG. 4A). The resulting protein should have contained all regions of ICP0 required for PML targeting and degradation (a homologous RING finger and the C-terminal targeting region) (16).

MeWo cells were either mock treated or transformed with constructs expressing ICP0, ORF61p or the fusion protein. Western blot analysis of cell lysates revealed that all protein products accumulated at similar levels and that addition of the C-terminal region of ICP0 did not alter ORF61p stability (FIG. 4B). The localization and abundance of these proteins and PML were then monitored. As described above, ICP0 expression led to disappearance of PML staining, whereas expression of ORF61p resulted in only slight changes in its intracellular staining pattern (FIGS. 2B and 2C). The fusion protein had a subcellular localization pattern distinct from that of ICP0 or ORF61p. In most cells (approximately 80-90%), the fusion protein was predominantly cytoplasmic (FIG. 4D). Similar to what was observed with the β-galactosidase fusion to this domain (18), a subpopulation of cytoplasmic PML containing bodies co-localized with the fusion protein. In the 10-20% of the population where the protein was nuclear, chromatin was marginated and the fusion protein filled the remaining nuclear space (FIG. 4E). However, in both cases, although PML distribution was altered, it was still detected. Thus, failure of ORF61p to lower intracellular levels of PML is an intrinsic property of the protein, and does not occur because ORF61p lacks an ND10 targeting domain.

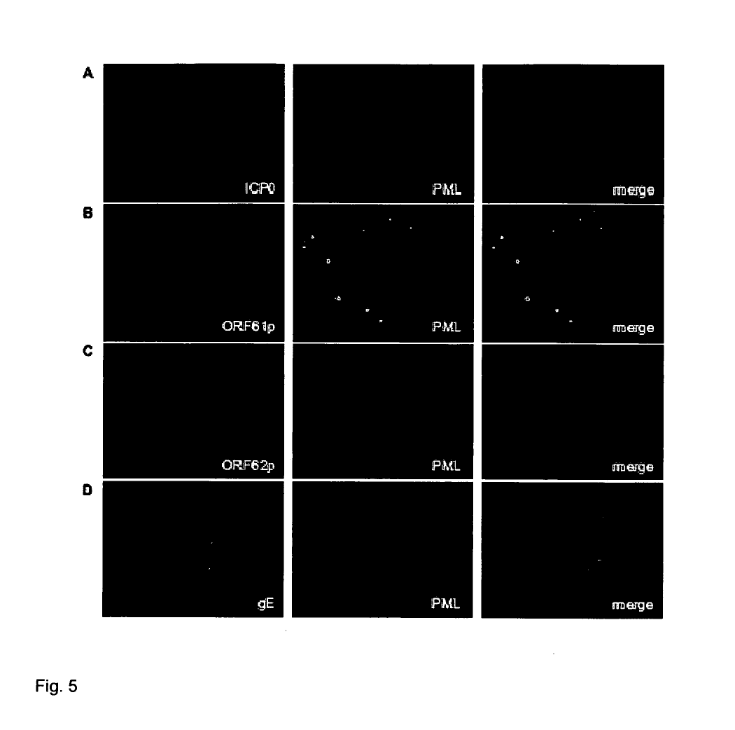

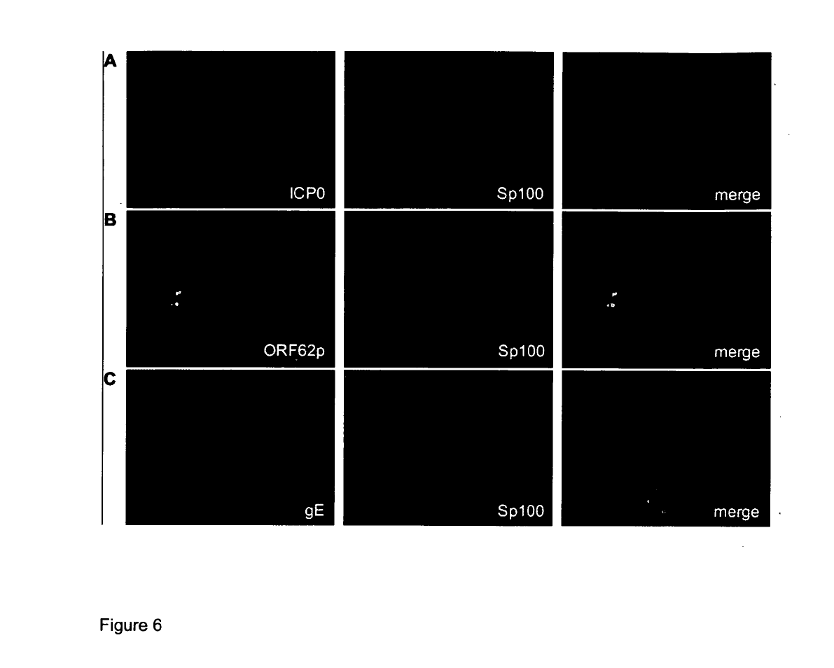

Distribution and abundance of PML and Sp100 during VZV infection. ORF61p alone does not efficiently degrade PML and Sp100 (FIGS. 2 and 3). To answer the question whether other VZV proteins affected distribution of these proteins during infection, MeWo cells were infected with HSV or cell free VZV and the intracellular distribution of viral and cellular proteins was monitored by immunofluorescence microscopy. As previously reported, in cells infected with HSV, staining for PML and Sp100 disappeared (FIGS. 5A and 6A). During the initial analyses, staining for ORF61p was the marker for virus infected cells. In cells expressing ORF61p, PML containing bodies appeared smaller in size and less bright when compared to uninfected cells. However, unlike in cells infected with HSV, PML was still detected in VZV infected cells (FIG. 5B). Nevertheless, because the expression kinetics of VZV encoded proteins is not fully understood, and it might be possible that other proteins expressed after ORF61p contribute to loss of PML during infection, ORF62p was used as an alternative marker for infected cells. This protein initially localizes to the nucleus of infected cells; however, it is subsequently phosphorylated and translocates to the cytoplasm later in infection (11). Therefore, its intracellular localization pattern is useful as a marker of infected cells and as an indicator of the stage of the virus replication cycle. In cells where ORF62p was nuclear both PML and Sp100 appeared as spherical structures, very similar to what is seen in uninfected cells (FIGS. 5C and 6B). Infected cells at late time points post infection were monitored using staining for the glycoprotein gE. Late in VZV infection, the localization of ND10 components was similar to what was observed in transformed cells (FIG. 2). Specifically PML bodies were still present though their abundance appeared to be decreased and their staining intensity was less than what was seen in uninfected cells (FIG. 5D). In contrast the characteristic punctate staining for Sp100 was not detected (FIG. 6C).

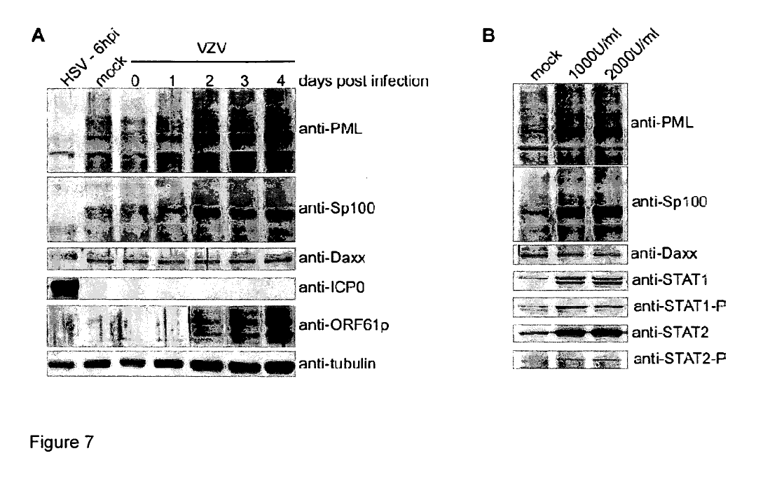

To further investigate the fate of ND10 components during VZV infection and quantitatively measure their abundance, MeWo cells were infected with either HSV or cell free VZV and virus and cell protein levels were monitored by western blotting. As previously described, HSV infection results in rapid degradation of multiple isoforms of PML and Sp100 (FIG. 7A) (8). VZV cell-free titers are low and the kinetics of virus replication are very slow compared to HSV. Therefore, to assay the effect of virus infection on these proteins, their levels were followed for several days post infection. In contrast to what occurs during HSV infection, levels of both PML and Sp100 increased during this period of observation (FIG. 7A). The rate of increase of PML and Sp100 was significantly higher than the rate of increase of tubulin, indicating that the increase in abundance of these proteins was not a result of cell growth and replication. To verify this, intracellular levels of Daxx, another constitutive component of ND10, was monitored. Unlike PML and Sp100, Daxx intracellular levels remained almost constant during the course of observation (FIG. 7A).

Because PML and Sp100 are induced by interferon (25, 60), this experiment attempted to answer the questions whether expression of these proteins is sensitive to interferon in MeWo cells, and whether interferon has an effect on Daxx. MeWo cells were treated with two different concentrations of interferon α. To verify that the interferon pathway was stimulated in MeWo cells, STAT1, STAT2 levels and their activated phosphorylated forms were monitored by western blotting (FIG. 7B). As evidenced by increased abundance and phosphorylation of these signaling molecules, the interferon response was active in MeWo cells. The abundance of the three ND10 components was measured. Although PML and Sp100 expression was induced, Daxx levels remained unaltered by interferon treatment (FIG. 7B).

These experiments suggest that increased levels of PML and Sp100 during VZV infection result from induction in response to interferon secreted by infected cells and failure of VZV to target these proteins for degradation.

The functions of PML, Sp100 and Daxx during VZV infection. ICP0 directed degradation of PML and Sp100 is beneficial for HSV replication (21, 22). Daxx restricts infection by HCMV and adenoviruses (34, 68, 72). Because VZV does not efficiently reduce levels of ND10 components, this experiment attempted to answer the question whether down-regulation of these proteins altered VZV replication kinetics and yields.

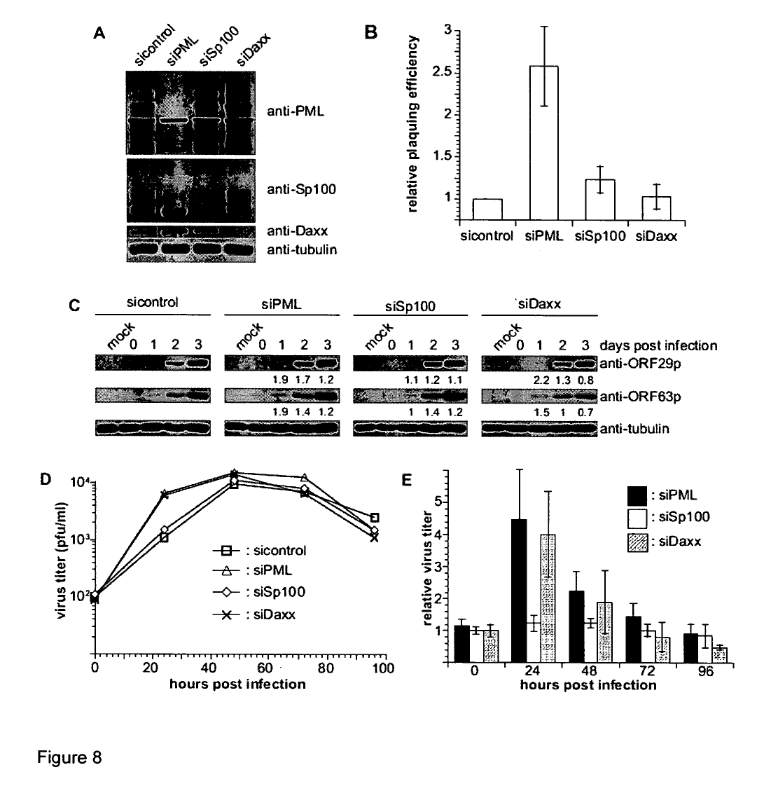

Recombinant retroviruses expressing siRNAs targeting either nothing, PML, Sp100 or Daxx mRNAs were used to transduce MeWo cells and generate stable cell lines (sicontrol, siPML, siSp100 and siDaxx respectively). The abundance and localization of the targeted proteins were monitored in these cell lines by western blot (FIG. 8A) and immunofluorescence microscopy (data not shown). As previously reported (21, 22), depletion of PML resulted in loss of integrity of ND10 bodies, a change in the expression pattern of Sp100, but no significant difference in Daxx levels. In contrast, down-regulation of Sp100 or Daxx did not alter either the levels or distribution of other ND10 components.

These cell lines were then used to measure VZV plaquing efficiency. Confluent monolayers were infected with serial dilutions of cell free virus stocks. Several days post infection monolayers were fixed and stained, and the plaques were counted. The number of plaques formed on each cell line was normalized to the number formed on control cells. The results revealed that when PML levels were reduced the number of plaques increased by approximately 2.5 fold (t₍ᵥₐₗ₎=0.0017) (FIG. 8B). This result mimicked what is seen with an ICP0⁻ mutant of HSV (22). In contrast, down-regulation of Sp100 resulted in a minor (1.2 fold), although consistent, increase in VZV titer (t₍ᵥₐₗ₎=0.031). Depletion of Daxx had no effect (FIG. 8B).

To further investigate the role of ND10 components on VZV growth, siRNA cell lines were infected with cell-free virus and accumulation of virus proteins was monitored over time (FIG. 8C). Band intensities corresponding to virus proteins in each depleted cell line were normalized to what was present in control cells at the same time point. The intracellular levels of ORF63p, an immediate early protein, and ORF29p, an early protein, were increased at early time points following infection of siPML and siDaxx cells. However, at late times the levels were similar to what was observed in control cells. Like the plaquing efficiency results, depletion of Sp100 had only a minor effect on virus protein levels.

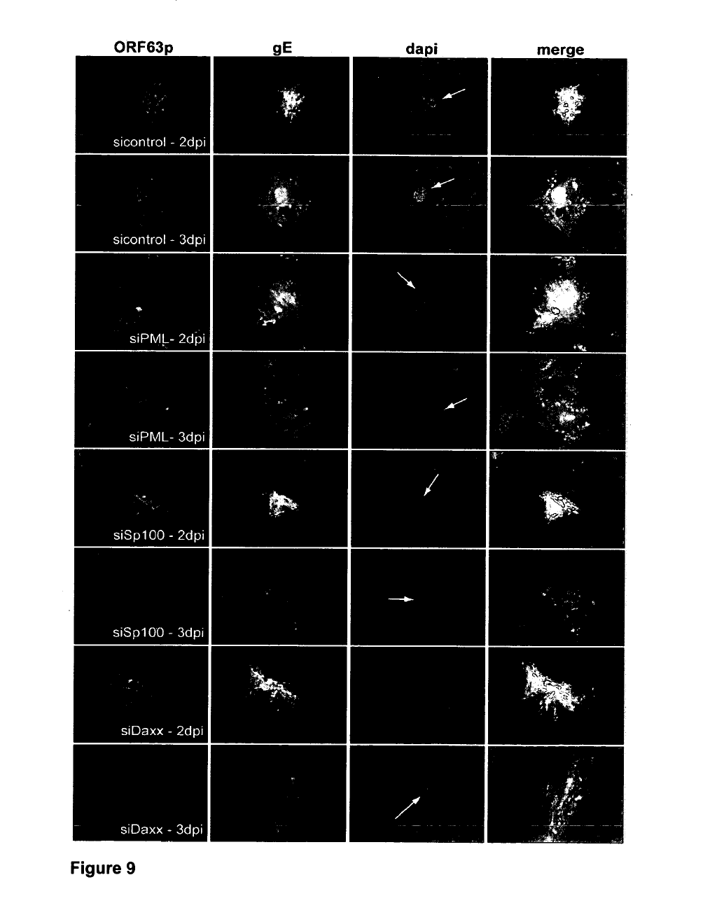

To study formation of infectious centers, siRNA cell lines were infected with cell-free VZV and at various times post infection cell-associated virus titers were measured. Consistent with the western analysis (FIG. 8C), the number of infectious centers formed in siPML and siDaxx cells increased early in infection before reaching a plateau similar to what occurred in sicontrol cells (FIGS. 8D and 8E). Depletion of Sp100 had little influence on infectious center yields (FIGS. 8D and 8E). Cytopathic effect (CPE) was more pronounced during infection of siPML cells and plaques were visible approximately 24 h earlier than in the other cell lines. In contrast, plaque size in siDaxx monolayers was considerably smaller compared to all other cell lines and virus induced CPE was minimal, even at late times in infection. Furthermore, although the protein accumulation and infectious center assays demonstrated accelerated virus replication similar to what occurred in siPML cells (FIG. 8C-E), surprisingly, the plaquing efficiency in siDaxx was identical to that in control cells (FIG. 8B). To probe the basis for these differences, siRNA cells grown on coverslips were infected, and at 2 and 3 dpi, cells were fixed and the expression of an immediate early protein (ORF63p) and a late glycoprotein (gE) were monitored by immunofluorescence microscopy (FIG. 9). The nuclei of control cells at 2 dpi formed the characteristic ring shaped structures that are indicative of cell fusion and efficient virus spread (37, 71). Infected foci in Sp100 depleted cells were similar in size to those formed in sicontrol cells. In contrast, VZV spread much faster in cells lacking PML or Daxx, as evidenced by formation of larger foci at 2 dpi. However, unlike siPML cells, where extensive fusion occurred, infection of siDaxx cells spread with no apparent CPE. Moreover, at 2 dpi, cells were only detached from the siPML monolayer, resulting in holes that scored as a plaque.

At 3 dpi (FIG. 9), CPE was obvious in monolayers from all cell lines except siDaxx. Extensive cell fusion was detected as evidenced by syncytia formation and the homogeneous staining pattern of viral proteins. In contrast to other cell lines, spread of infection in siDaxx cells was different. Although VZV spread to infect neighboring cells, individual intact infected cells were detected without any evidence of cell fusion. This morphology was strikingly different from sicontrol cells. Importantly, cells were not detached even at this late stage in virus infection, which explains the considerably smaller plaque size. Importantly, because infected cells remained in the monolayer and failed to round up, they were often not scored as plaques in the siDaxx line, resulting in a seemingly lower plaquing efficiency (FIG. 8B).

This analysis of virus replication in cells lacking the major components of ND10 bodies demonstrated that PML is a repressor of wild-type VZV growth, whereas Sp100 had little if any role in this process. Daxx appeared to have a distinct function, because although silencing of this protein initially resulted in accelerated replication and protein accumulation, virus directed syncytia formation was defective.

### Discussion

Components of ND10 bodies, including PML, Sp100 and Daxx, associate with DNA virus genomes and contribute to an intrinsic antiviral mechanism that acts to repress expression from these genomes (10, 21, 22, 32, 35, 47, 67-69). Herpesviruses have evolved countermeasures that bypass this cellular repression mechanism to ensure their efficient replication and spread. HSV encodes a potent transcriptional activator, ICP0, that targets ND10 associated proteins for proteasomal degradation, resulting in increased expression of immediate early virus genes (21, 22).

VZV, a closely related alphaherpesvirus, encodes ORF61p, an ICP0 ortholog. Previous studies have emphasized the conservation of biological activities between these two proteins and have demonstrated that both are activators of gene expression (49, 50). However, it has also previously been shown that unlike ICP0, ORF61p fails to overcome a requirement for the co-chaperone protein BAG3 during virus replication, suggesting that the orthologs have diverse functions (38).

Amino acid sequence alignment of these proteins revealed that while ORF61p retains the conserved RING finger residues of ICP0, it lacks the C-terminus of its HSV ortholog (FIG. 1). Both of these regions are required for efficient targeting of ICP0 to ND10 bodies and degradation of their components (16). ORF61p's lack of ICP0's C-terminus and its associated ND10 targeting domain raised the possibility that ORF61p is unable to degrade components of PML bodies. Immunofluorescence analysis of cells transiently expressing ICP0 or ORF61p and western blot analysis of proteins from cells infected with recombinant adenoviruses expressing the herpesvirus proteins revealed that ORF61p does not deplete PML and that Sp100 levels are decreased much less efficiently than in cells expressing ICP0 (FIG. 3). Furthermore, attempts to target ORF61p to ND10 by addition of the ICP0 targeting domain did not change this phenotype (FIG. 4).

Because ICP0 contains two separate E3 ubiquitin ligase activities, it was described as a two-headed ubiquitin ligase (reviewed in (30)) (FIG. 1). Herpes simplex virus ubiquitin ligase (HUL)-1 is encoded by exon 3 of ICP0 and is responsible for degradation of cdc34 (28, 29, 31). However, the HUL-2 activity that promotes degradation of PML and Sp100 maps to the RING finger domain of ICP0 (4, 31). RING domains in the appropriate molecular context have been implicated in proteasomal degradation (42). Importantly, binding of an ubiquitin protease (HAUSP-USP7) to the C-terminus of ICP0 was suggested to promote degradation of RING finger substrates (30). This binding might result in sequestration of USP7 from newly ubiquitinated HUL-2 substrates and ensure their efficient targeting for proteasomal degradation (5, 20, 30).

Based on these observations, two scenarios are envisioned that explain why ORF61p does not cause disappearance of PML and Sp100. Although the RING finger domain is required for its transcriptional activation activity (48), the molecular context of the rest of ORF61p might be inappropriate for it to act as an E3 ubiquitin ligase. Alternatively, lack of an ubiquitin specific protease binding site within ORF61p might lead to availability of USP7, rapid de-ubiquitination of its targets, and thus protection from proteasomal degradation. The latter hypothesis is favored, as accumulating evidence suggests that unlike the self preservation properties of ICP0, which depend on binding of USP7 (5), ORF61p is rapidly degraded in a proteasomal dependent manner that requires a functional RING finger domain (Kyratsous, DeLong and Silverstein, unpublished). This observation implies that the RING finger of ORF61p possesses E3 ligase activity and can drive auto-ubiquitination, however, lack of a protease binding site results in its depletion. In support of this, amino acids within the sequence of ICP0 that are required for binding of USP7 (19) are not found in ORF61p (FIG. 1). Upon further analysis of the relationship between VZV proteins and components of ND10s, it was observed that, unlike what occurs during HSV infection, the abundance of PML and Sp100 increases during VZV infection (FIG. 7A). This increase is specific for interferon-stimulated components of ND10s (FIG. 7B), as Daxx levels do not change throughout the course of infection (FIG. 7A). Because VZV induces interferon, it is believed that virus proteins do not directly induce synthesis of ND10 components, and that therefore, the increase in abundance of PML and Sp100 is an indirect effect of interferon stimulation. The possibility that other pathways not related to interferon may contribute to increased levels of PML and Sp100 in infected cells cannot be excluded.

Surprisingly, although autonomous expression of ORF61p results in decreased levels of Sp100 (FIG. 3), VZV infection results in an increase (FIG. 7). It is posited that induction by interferon during infection masks degradation of Sp100 by ORF61p. This contrasts with what is observed during HSV infection, but remains consistent with our observation that ICP0 causes a more efficient decrease of Sp100 when compared to ORF61p (FIG. 3).

HSV mutants lacking ICP0 are hypersensitive to interferon (51) and this effect is mediated by PML (7). In contrast, although VZV is sensitive to interferon, ORF61 mutants, unlike ICP0 mutants, are not hypersensitive to interferon (1). These data, along with the observation that PML is not degraded during VZV infection, suggest that interferon inhibits replication of these two human alphaherpesviruses by distinct mechanisms and that these viruses have evolved different and specific countermeasures. As a result, in contrast to HSV, it is likely that VZV does not require degradation of PML to overcome inhibition by interferon. PML, Sp100 and Daxx were reported to suppress the early stages of herpesvirus replication (21, 22, 67-69). Stable cell lines depleted of each of these proteins were used to analyze whether these proteins also affect the replication kinetics and yield of VZV (FIG. 8A). Unlike its role in HSV infection, Sp100 had little effect on plaquing efficiency, gene expression and infectious center titer in cells infected with VZV. However, infection of both siPML and siDaxx cell lines resulted in an increase in titer and accumulation of virus proteins at early times. Thus, these proteins specifically inhibit the early stages of virus replication. Despite these differences, cell-associated titers of VZV reached the same peak titer at later times in infection in all cell lines. It is posited that VZV replication is controlled by two independent host mediated steps: an early block that is mediated by PML, Daxx and possibly other host proteins, and a late block that determines virus yield. Although depletion of proteins that function early to inhibit the initial stages of the virus life cycle results in accelerated replication kinetics, it is not sufficient to increase spread and development of infectious centers.

The function of ND10 components during wild-type HSV infection is difficult to study, as these proteins are rapidly degraded after ICP0 expression and are absent from infected cells. Thus, depletion of these host products has no effect on wild-type virus replication kinetics and yield, but enhances replication of an ICP0⁻ virus that fails to direct degradation (21, 22). The results of this study demonstrate that wild-type VZV mimics ICP0⁻ virus replication in PML depleted cells (FIG. 8) and fails to direct degradation of the major component of ND10 bodies (FIG. 3). In contrast to PML, Sp100 abundance is partially decreased when ORF61p is expressed (FIG. 3) and depletion of the protein has only a minor effect on virus replication (FIG. 8). Thus, VZV might have evolved to titrate Sp100 levels to the extent required for efficient replication. Alternatively, the small amounts of Sp100 remaining within the cells after siRNA depletion might be sufficient for it to silence VZV.

In conclusion, this study shows that VZV grown in cell culture behaves as a unique member of the alphaherpesvirus family. Unlike other ICP0 orthologs (55), ORF61p does not direct degradation of ND10 components. VZV's interaction with this intrinsic defense mechanism is most similar to how adenoviruses deal with host mediated silencing (72, 73). Neither virus is able to degrade ND10 proteins and yet both overcome restriction by interferon. Moreover, in contrast to HSV but in kind with adenoviruses, VZV replication is unaffected by depletion of Sp100, but its replication is accelerated when PML and Daxx are silenced. Although HSV and VZV are considered to be very similar, this study demonstrates that they have evolved unique and specialized ways to interfere with host cell repression to ensure their efficient growth and spread.

### Results II

Alphaherpesviruses encode orthologs of the herpes simplex virus (HSV) α gene product ICP0. ICP0 is a nuclear phosphoprotein that behaves as a promiscuous activator of viral and cellular genes (83, 87, 104, 105). ICP0 also functions as an E3 ubiquitin ligase to target several host proteins for proteasomal degradation (80, 86, 87, 92, 102). Through this activity, ICP0 promotes degradation of components of nuclear domain 10 (ND10) bodies, including the promyelocytic leukemia (PML) protein and Sp100. These proteins are implicated in silencing of herpesvirus genomes (86, 87, 98, 110). Therefore, ICP0 mediated degradation of ND10 components may disrupt silencing of HSV genes in order to enable efficient gene expression. This hypothesis provides a plausible mechanistic explanation of how ICP0 induces gene activation.

Introduction of DNA encoding the ICP0 orthologs from HSV, bovine herpes virus, equine herpes virus and varicella zoster virus (VZV) can also affect nuclear structures and proteins (103). In addition and more specific to this report, ORF61p, the VZV ortholog, activates viral promoters and enhances infectivity of viral DNA, as does ICP0, the prototype for this gene family (100, 101). However, two key biological differences between the HSV and VZV orthologs have previously been demonstrated. Unlike ICP0, ORF61p is unable to complement depletion of BAG3, a host co-chaperone protein. As a result, VZV is affected by silencing of BAG3 (91), whereas growth of HSV is only altered when ICP0 is not expressed (93). Furthermore, while both proteins target components of ND10s, expression of ICP0 results in degradation of both PML and Sp100, whereas ORF61p specifically reduces Sp100 levels (92). These findings suggest that these proteins have evolved separately to provide different functions for virus replication.

Virus mutants lacking the ICP0 gene have an increased particle to plaque forming unit (pfu) ratio, substantially lower yield and decreased levels of α gene expression, in a multiplicity of infection (moi) and cell type dependent manner (78, 80, 84, 109). These mutants are also defective at degrading ND10 components (99). Depletion of PML and Sp100 accelerates virus gene expression and increases plaquing efficiency of HSV ICP0 defective viruses, but has no effect on wild-type virus, suggesting that PML and Sp100 are components of an intrinsic anti-HSV defense mechanism that is counteracted by ICP0's E3 ligase activity (86, 87). Interestingly, ICP0 null viruses are also hypersensitive to interferon (IFN) (102), a property that was suggested to be mediated via PML (79).

An HSV mutant virus that expresses ORF61p in place of ICP0 was constructed in order to directly compare the activities of the two orthologs. The resulting chimeric virus only partially rescues the ICP0 null phenotype. Such studies emphasize the biological differences between ICP0 and ORF61p and shed light on the requirements for PML and Sp100 during infection.

Materials and Methods: Mammalian cells. Human melanoma (MeWo), siPML (93), siSp100 (92), L7 (106) and U2OS cells were maintained as previously described (91, 111).

DNA transformation. DNAs were transformed into the appropriate cell lines using Fugene HD [Roche, Indianapolis, Ind.].

Drug treatment. Interferon α was purchased from PBL Biomedical [Piscataway, N.J.].

Viruses. [i] HSV. Strains used were wild-type HSV-1 (Glasgow strain 17) and an ICP0 null virus derivative of strain 17 (dl1403) (109). [ii] HSV expressing VZV ORF61p (HSV-ORF61). dl1403 nucleocapsids were co-transfected with linearized pCPC-061 into MeWo cells. Large plaques were picked and screened for recombinant viruses by PCR. Plaques that were positive for ORF61p but not for ICP0 coding sequence were plaque purified five times.

Virus growth assays. [i] Plaque assays. Confluent monolayers of MeWo, siPML, siSp100, L7 or U2OS cells were infected with 10-fold serial dilutions of virus stocks and the monolayers were fixed and stained, and plaques were counted. [ii] Growth curves. The titers of all HSV stocks were determined prior to analysis by titration on the ICP0-complementing cell line L7. Virus yield was determined as previously described (93).

Hirt DNA extraction. Hirt DNA was prepared as described (90).

Plasmid construction. VZV ORF61 was PCR amplified from VZV genomic DNA (Jones strain) using RV61 (5′-GGGTCGACTTGCATTACCCTATCCCAGTATT-3′) (SEQ ID NO:7) and 3′Sal61 (5′-CCGTCGACCCCAACAAACTAGGACTTCT-3′) (SEQ ID NO:8). The PCR product was cloned in pCR2.1-TOPO to generate pCPC-T61cJ. The ORF61 coding sequence was excised as an NcoI/SalI fragment that was used to replace sequences encoding ICP0 in NcoI/SalI digested pDS17 (113), to yield pCPC-061.

All primers were obtained from Operon Biotechnologies [Huntsville, Ala.] and all vector inserts were verified by DNA sequencing.

Analysis of recombinant virus genomes. Hirt DNAs were interrogated for the presence of ORF61 sequences and the absence of IE-0 coding sequences by performing PCR using the primers: 0 for: 5′-ACAGAAGCCCCGCCTACGTT-3′, 0rev: 5′-GGTGCCCGTGTCTTTCACTTTTC-3′, 61for: 5′-GGGAATTCGGGGCCCCTTCAATCGTCGGCTAG-3′, 61rev: 5′-TGCGGCCGCGAATCTCGCGTTTCCCTCTGTTCC-3′ (SEQ ID NOS:3-6, respectively).

Antibodies. Polyclonal antibodies to ICP0 were described (20). Monoclonal antibodies to ICP0 and ICP4 were purchased from the Rumbaugh-Goodwin Institute [Plantation, Fla.]. Polyclonal antibodies against ORF61p were described (92). Monoclonal antibodies to tubulin were obtained from Santa Cruz Biotechnology [Santa Cruz, Calif.]. Polyclonal antibodies against PML and Sp100 were purchased from Chemicon [Temecula, Calif.]. Goat anti-rabbit and anti-mouse antibodies conjugated to horseradish peroxidase for immunoblotting were obtained from KPL [Gaithersburg, Md.].

SDS-PAGE and western blotting. Infected or biochemically transformed cells were washed twice with cold PBS, lysed in 1.5×SDS sample buffer [75 mM TrisHCl pH 6.8, 150 mM DTT, 3% SDS, 0.15% bromophenol blue, 15% glycerol], and boiled. Host and viral proteins were subjected to SDS-PAGE (94). Proteins were transferred to nitrocellulose membranes before western blotting. After blocking membranes in 5% non-fat milk in PBST, immobilized proteins were reacted with the appropriate antibodies in 1% nonfat milk in PBST. Membranes were washed three times for 5 min each with PBST, incubated with an anti-rabbit or anti-mouse antibody conjugated to horseradish peroxidase, and washed again three times for 5 min with PBST and twice with PBS. Antibodies were visualized by addition of LumiGLO substrate [KPL] and exposure to X-ray film.

### Results

Generation of a VZV-HSV recombinant expressing ORF61p. Coinfection with VZV complemented growth of an HSV-ICP0 mutant (109). Subsequently, a cell line that conditionally expressed ORF61p was used to complement an ICP0 null mutant (24). This latter experiment suggested that these virus orthologs shared some biological activities. However, these proteins differentially affected ND10 components, and wild-type VZV, but not HSV, showed a distinct requirement for these components (92). Therefore, to further dissect the function of ORF61p, this study explored if it might substitute for HSV ICP0 when it replaced the duplicated immediate early 0 (IE-0) loci.

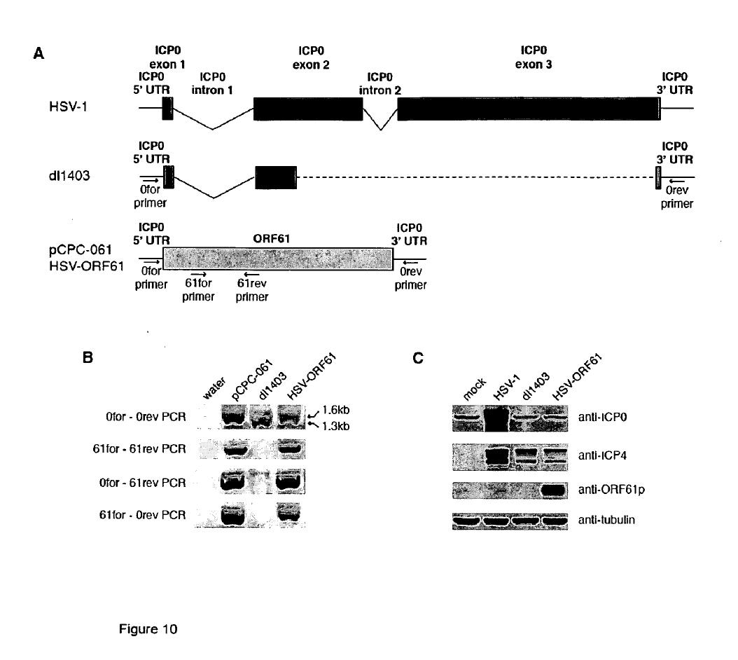

To replace the loci encoding ICP0 HSV, dl1403 was used as the viral backbone. dl1403 encodes the first 105 aa and an additional 56 aa that are derived from an out of frame fusion of the second and third exons of the IE-0 gene. ORF61p coding sequences were amplified and inserted in an NcoI/SalI digested ICP0 clone as described in Materials and Methods. The NcoI site encompasses the AUG codon used by both genes to initiate synthesis of their respective proteins. The structural integrity of the resulting plasmid (pCPC-061), that retains the IE-0 promoter and 3′UTR, was verified by restriction endonuclease cleavage and DNA sequence analysis. Subsequently, pCPC-061 was linearized and co-transfected into MeWo cells with dl1403 nucleocapsids (107). The resulting recombinant virus was titrated on MeWo cells and large plaques were picked with the presumption that expression of ORF61p would complement the ICP0-defect (100, 109). Hirt DNAs prepared from these plaques were screened by PCR with primers upstream and downstream of IE-0 and two internal primers homologous to ORF61 (FIG. 10A). Plaques containing virus DNA with sequences encoding ORF61p and lacking DNA encoding ICP0 (FIG. 10B) were further purified and used to infect cells to determine if they expressed ORF61p. The results of this analysis are shown in FIG. 10C and are summarized as follows: western blot analysis of cells infected with dl1403 or HSV-ORF61 demonstrated that they expressed similar amounts of ICP4 at 6 hr post infection and no ICP0 and that HSV-ORF61 expressed ORF61p. Thus, in HSV-ORF61 both copies of a defective IE-0 gene were replaced with ORF61p coding sequences and the resulting virus expressed ORF61p under control of the IE-0 promoter.

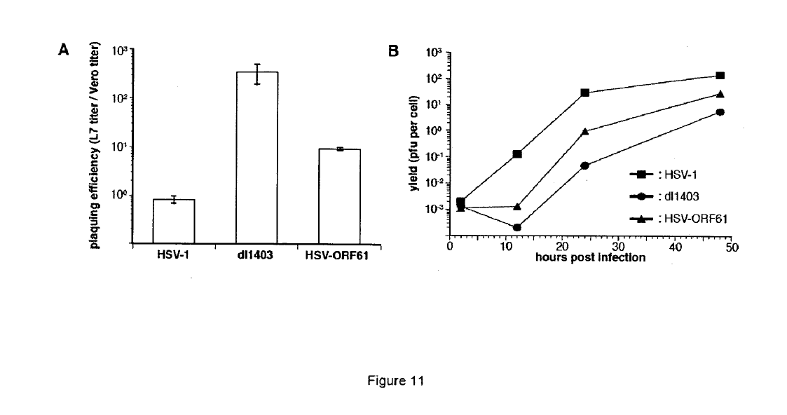

Growth and plaguing efficiency of HSV-ORF61. Two experiments were done to test whether expression of ORF61p rescued the ICP0 null phenotype. First, wild-type, dl1403 and HSV-ORF61 were titrated on L7 and Vero cells and relative plaquing efficiencies were calculated as a percentage of the titer on L7 cells versus the titer on Vero cells. HSV dl1403, and other ICP0 mutant viruses, have a high particle/pfu ratio that is evident when their titer is measured on complementing cells such as L7 and compared to their titer on the parental Vero cell line. The plaquing efficiencies of the three viruses were: wild-type=0.9, dl1403=340 and HSV-ORF61=9.5 (FIG. 11A). Thus, although expression of ORF61p enhanced the plaquing efficiency of HSV-ORF61 over dl1403 by approximately 35-fold, it was not sufficient to restore wild-type plaque formation. This study next investigated how expression of ORF61p affected the growth kinetics of the recombinant virus in MeWo cells. Cells were infected at a low moi, samples were harvested over time, and the yield of infectious virus per cell was determined by plaque assay on L7 cells. Analysis of the growth curves revealed that HSV-ORF61 replicated with kinetics that were intermediate between wild-type and dl1403 (FIG. 11B). These two experiments led to the conclusion that, in terms of plaguing efficiency and virus yield, ORF61p could not fully compensate for lack of ICP0.

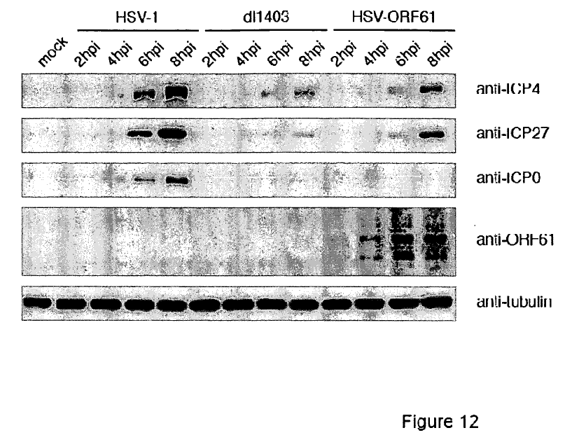

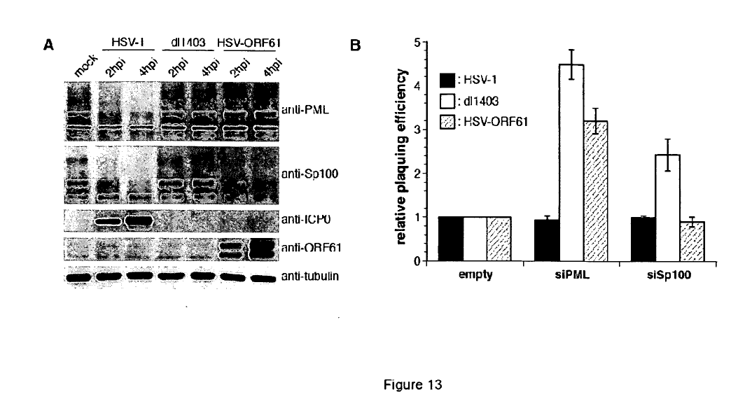

Accumulation of virus specified proteins in cells infected with HSV-ORF61. Growth defects of ICP0 mutants at low mois manifest as delayed expression and decreased accumulation of all classes of virus-specified proteins (2, 4). Therefore, MeWo cells were infected with wild-type, dl1403 and HSV-ORF61 at a moi of 0.2. Immunofluorescence analysis for ICP4 (data not shown) was used to verify that each virus infected equal numbers of cells. Cell lysates were prepared at the indicated times and processed for western blot analysis. Analysis of protein abundance and the kinetics of synthesis revealed that under this condition of low moi, accumulation of ICP4 was detected in cells infected with wild-type virus at 4 hpi. In contrast, this protein was not detected in cells infected with either dl1403 or HSV-ORF61 until 6 hpi (FIG. 12). Furthermore, ICP4 abundance at 8 hpi was significantly decreased in cells infected with mutant viruses compared to wild-type infected cells. As previously described, the kinetics of synthesis and accumulation of ICP27 depends on expression of functional ICP0 (97). This phenotype was only partially reversed when ORF61p was expressed (FIG. 13). As expected, ICP0 and ORF61p were only detected in cells infected with viruses that expressed these proteins. These data, consistent with studies on plaquing efficiency and growth (FIG. 11), demonstrated that ORF61p did not fully phenocopy the biological properties of ICP0, although it clearly boosted replication of an ICP0-virus.

Fate of ND10 components following infection. ICP0 is necessary and sufficient to dissociate ND10s and target their two major components, PML and Sp100, for proteasomal degradation. In contrast, ORF61p does not degrade PML but decreases Sp100 levels (92). Accordingly, the fate of PML and Sp100 was followed during an infection with HSV-ORF61 and compared with what occurred in cells infected with wild-type or dl1403. Western blot analysis revealed that degradation of PML was detected in cells infected with wild-type virus as early as 2 hpi. In contrast, PML levels were not altered in cells infected with either dl1403 or HSV-ORF61 (FIG. 13A). These results corroborated previous findings and demonstrated that ORF61 did not affect the steady state level of PML even when other HSV immediate early or early proteins were present. Sp100 is another major component of ND10s and it is well known that it is efficiently degraded following infection with HSV in an ICP0 dependent fashion (80). This study demonstrated that in the context of HSV gene expression, ORF61p effectively directed degradation of Sp100 (FIG. 13A).

Wild-type HSV and VZV viruses are differentially affected by depletion of PML or Sp100 (92). Relative plaquing efficiency of HSV-ORF61 in siPML and siSp100 cells was measured and compared to the efficiency of wild-type HSV and dl1403 in order to determine if HSV-ORF61 was affected by the down regulation of the host proteins PML or Sp100. As previously reported, wild-type virus plaquing efficiency was not affected when titrated on cells depleted for PML (siPML) or Sp100 (siSp100) (FIG. 13B). In contrast, dl1403 was partially complemented in the absence of these ND10 components (FIG. 13B). The relative plaquing efficiency of HSV-ORF61 phenocopied VZV (92). More specifically, virus titer increased in siPML cells, whereas it remained unchanged in siSp100 cells (FIG. 13B).

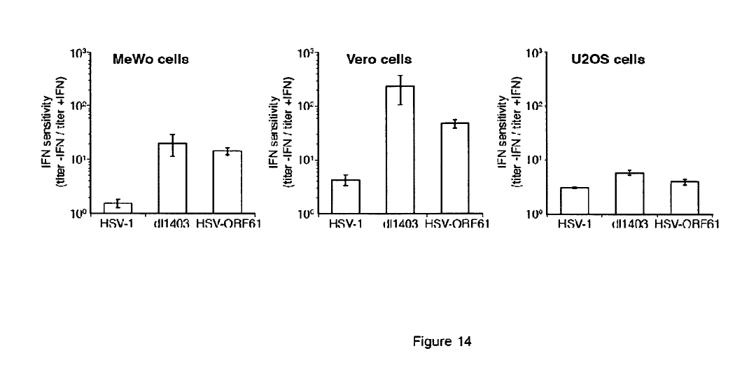

Effect of interferon on virus replication. Previous studies suggested that HSV's interferon (IFN) sensitivity is mediated via PML and proposed that an ICP0-virus is hypersensitive in part because it fails to degrade this cellular protein. Having shown that HSV-ORF61 was unable to degrade PML, this study investigated how IFN treatment would affect the growth of this mutant virus. A comparison was done of the plaquing efficiency of HSV-ORF61p, wild-type HSV-1 and dl1403 on MeWo, Vero (which respond to but do not express IFN (82)) and U2OS (a cell line that complements ICP0 mutant viruses (112)) cells in the presence and absence of IFN. The plaquing efficiency of both dl1403 and HSV-ORF61 in MeWo and Vero cells was affected by interferon-stimulated genes (ISGs) presumably synthesized in response to IFN (FIG. 14). The small difference (4 to 5-fold) in sensitivity seen with wild-type virus on MeWo and Vero cells was not a result of differences in absolute plaquing efficiency but rather reflected greater sensitivity of all viruses to the effects of IFN in Vero cells (Table 1).

TABLE 1
Virus titers

| | HSV-17 | dl1403 | HSV-ORF61 |
|---|---|---|---|
| MeWo −IFNα | 1.65 × 10⁸ +/− 7.07 × 10⁶ | 2.55 × 10⁴ +/− 1.63 × 10⁴ | 1.70 × 10⁷ +/− 1.41 × 10⁶ |
| MeWo | 1.10 × 10⁸ +/− 1.41 × 10⁷ | 1.20 × 10³ +/− 2.83 × 10² | 1.20 × 10⁶ +/− 2.83 × 10⁵ |
| +IFNα | | | |
| Vero −IFNα | 1.30 × 10⁸ +/− 2.83 × 10⁷ | 7.00 × 10⁴ +/− 8.49 × 10⁴ | 7.75 × 10⁶ +/− 1.77 × 10⁶ |
| Vero +IFNα | 3.00 × 10⁷ | 4.65 × 10² +/− 6.15 × 10² | 1.65 × 10⁵ +/− 7.07 × 10³ |
| U2OS −IFNα | 1.23 × 10⁸ +/− 3.89 × 10⁷ | 4.50 × 10⁷ +/− 7.07 × 10⁶ | 4.15 × 10⁷ +/− 3.32 × 10⁷ |
| U2OS | 4.00 × 10⁷ +/− 1.41 × 10⁷ | 7.75 × 10⁶ +/− 3.54 × 10⁵ | 1.00 × 10⁷ +/− 7.07 × 10⁶ |
| +IFNα | | | |

In support of this is the increased sensitivity of dl1403 to IFN in Vero cells. As previously described (102), U2OS cells rescued the sensitivity of ICP0 mutants to IFN. In a similar fashion, plaquing efficiency of HSV-ORF61 following treatment with IFN was also rescued (FIG. 14). These analyses revealed that while VZV ORF61p substituted for some of ICP0's functions it was clear that it did not complement all of the defects in dl1403 as evidenced by HSV-ORF61's failure to recapitulate the wild-type IFN resistant phenotype (FIG. 14).

### Discussion

HSV ICP0 is a RING finger protein that acts as a strong and promiscuous transcriptional activator of gene expression. Orthologs of ICP0 exist in other members of the alphaherpesvirus family. These proteins are related to ICP0 by virtue of their location within the virus genome and ability to influence gene expression. Sequence similarities are limited, with the exception of a RING finger close to the N-termini in all orthologs. Specifically, the ICP0 ortholog in VZV, ORF61p, accelerates replication of an ICP0-virus when co-expressed and also influences gene expression (100,101). In spite of these similarities, the lack of homologous ICP0 sequences within the ORF61 gene has previously been emphasized, and a suggestion that these proteins have diverse functions (92) has been made.

This study compares the activities of ICP0 and ORF61p by constructing a mutant HSV virus that expresses ORF61p in place of ICP0 under control of the ICP0 promoter and 3′UTR. Therefore, the orthologs are expressed in an identical genetic background and any difference observed in biological activities of the two viruses should be solely a result of which virus protein is expressed.

Comparison of the growth and protein expression profiles during infection with wild type, ICP0- and HSV-ORF61 demonstrated that although ORF61p partially rescues the ICP0 null phenotype, replication of HSV-ORF61 is less efficient than replication of wild-type virus. There is a possibility that these proteins are expressed to different levels, have distinct half-lives and interact with HSV proteins differently, and that this might affect the growth phenotype of the HSV-ORF61. The conclusion is that ORF61p, as expressed, lacks some of ICP0's functions.

ICP0 expressed from an adenovirus caused efficient depletion of two major ND10 components, PML and Sp100, whereas an ORF61p expressing adenovirus reduced only Sp100 levels. Here, the effect of these proteins on ND10 component abundance during virus replication was compared. It was observed that even when other HSV proteins were expressed, ORF61p specifically decreased Sp100 with no effect on PML.

ND10s have been suggested to provide a nuclear form of innate immunity. Specifically, ND10 components act to repress expression of herpesvirus and other DNA virus genomes. In that vein it is interesting that replication and plaguing efficiency of dl1403 but not wild-type virus are augmented in cells that lack PML or Sp100 (FIG. 14) (85,86). In contrast, replication and plaguing efficiency of wild-type VZV is unaffected by depletion of Sp100 and augmented in siPML cells (92). Based on these observations and assuming that only ICP0 and ORF61p are necessary for the observed plaguing efficiencies in the different cell lines, one might expect that an HSV virus expressing ORF61p in place of ICP0 would phenocopy VZV. Therefore, the plaguing efficiency of HSV-ORF61 on siPML and siSp100 cells were compared to their parental control cell lines. Although virus replication was partially complemented when PML was depleted, reduction of Sp100 levels did not affect virus replication. This was a further demonstration that ORF61p has evolved to titrate Sp100 levels to allow efficient virus replication. However, unlike ICP0, ORF61p does not target PML; therefore, when only ORF61p is expressed, the PML host protein remains available to repress virus growth and replication.

Viruses expressing ICP0 or ORF61 differentially affect Sp100 degradation. Sp100 is normally resolved as three species during SDS electrophoresis (89,108). The species recognized by the study antibody in terms of rate of electrophoretic migration were Sp100A, Sp100A-SUMO and Sp100-HMG. In cells infected with wild-type virus the higher molecular weight species of Sp100 gradually disappeared whereas Sp100A was stabilized (FIG. 14). This electrophoretic pattern of Sp100 mimicked what was observed in cells depleted of PML by siRNA (85,92). In contrast, when ORF61p was expressed in place of ICP0, all isoforms and modified species of Sp100 gradually disappeared during infection, with no apparent difference in PML's electrophoretic pattern. This is in agreement with siRNA experiments, which show that, unlike depletion of PML, down-regulation of Sp100 has no effect on other components of ND10 bodies (85, 86, 92). Furthermore, it has previously been observed that infection of cells with an adenovirus expressing ICP0 resulted in disappearance of PML and Sp100 species except for Sp100A, whereas an ORF61p expressing adenovirus reduced all Sp100 forms with no effect on PML (92).

These results lead to the proposition that while these alphaherpesvirus orthologs target components of ND10, they do so in distinct ways. HSV ICP0 targets PML for proteasomal degradation. Reduction of PML levels results in disappearance of Sp100 species, except Sp100A. Therefore, by targeting PML, ICP0 directly or indirectly targets both major ND10 components. In contrast, ORF61p independently targets Sp100 for degradation. Unlike with ICP0, reduction of Sp100 levels has no apparent effect on other ND10 proteins. Differential targeting of ND10 proteins by these orthologs may account for at least some of the observed differences in their biological activities.

The precise role of PML during virus infection remains elusive. It is known however that HSV mutants lacking ICP0 and VZV mutants lacking ORF63p are hypersensitive to interferon (102) and that this effect is mediated by PML (79). In contrast, wild-type HSV (102) and VZV (77) are less sensitive to IFN. These data, along with the observation that PML is not degraded during VZV infection (92), suggest that interferon inhibits replication of these two human alphaherpesviruses by distinct mechanisms, and that these viruses have evolved different and specific countermeasures. As a result, in contrast to HSV, it is likely that VZV does not require degradation of PML to overcome inhibition by interferon. These studies provide a basis for a molecular understanding of the functional differences between HSV ICP0 and VZV ORF61p. Unpublished observations are consistent with previous reports that, in spite of its functional handicap, ORF61p still activates both VZV and HSV promoters. However, as noted above, ORF61p lacks the immune regulatory activities of ICP0. Based on these observations it is likely that Sp100 depletion is required to boost virus gene expression, whereas the activities that affect commandeering of the interferon arm of the innate immune system might be solely mediated via PML. Therefore, further dissection of the functions of these orthologs might provide insight into the role of ND10 components during infection.

These results are important regarding HSV backbones as vaccine vectors (19). Expression of ICP0 is required for chromatin modification and remodeling to allow efficient expression of virus genes (81, 88, 95). However, expression of ICP0 interferes with innate immunity. Moreover, deletion of ICP0 results in decreased virus titer. Thus, use of HSV-ORF61 as the basis for a backbone in place of ICP0 provides an advantageous alternative to current herpesvirus based vectors.

### Results III

Alphaherpesviruses encode orthologs of the HSV α gene product ICP0. ICP0 is a nuclear phosphoprotein that behaves as a promiscuous activator of viral and cellular genes and also functions as an E3 ubiquitin ligase to target host proteins for proteasomal degradation. Through this activity, ICP0 promotes degradation of components of ND10 bodies, including PML and Sp100. These proteins are implicated in silencing of herpesvirus genomes. Therefore, ICP0 mediated degradation of ND10 components may disrupt silencing of HSV genes to enable efficient gene expression. This hypothesis provides a plausible mechanistic explanation of how ICP0 induces gene activation. ORF61p, the VZV ortholog, activates viral promoters and enhances infectivity of viral DNA like ICP0. However, two key biological differences between the HSV and VZV orthologs have been demonstrated. First, unlike ICP0, ORF61p is unable to complement depletion of BAG3 a host co-chaperone protein. As a result, VZV is affected by silencing of BAG3, whereas growth of HSV is only altered when ICP0 is not expressed. Furthermore, while both proteins targeted components of ND10s, expression of ICP0 resulted in degradation of both PML and Sp100, whereas ORF61p specifically reduced Sp100 levels. Thus these proteins have evolved separately to provide different functions for virus replication. To directly compare these orthologs an HSV mutant virus was constructed that expressed ORF61p in place of ICP0 (FIG. 10).

In the accompanying graph (FIG. 11b) it is demonstrated that while ORF61p restored robust growth that is lacking from an ICP0-virus (dl1403) the resulting chimeric virus only partially rescued the ICP0 null phenotype. This finding demonstrated that there are profound biological differences between ICP0 and ORF61p. More importantly, when considering a VZV vaccine it is necessary to recall that VZV but not HSV is sensitive to interferon (IFN). Therefore, HSV-ORF61 was tested for its ability to degrade PML and it was demonstrated that it, like wild-type VZV failed to degrade. Accordingly, it was asked if IFN treatment affected growth of this mutant virus and demonstrated that plaquing efficiency of both dl1403 and HSV-ORF61 in MeWo and Vero cells was decreased by ISGs presumably synthesized in response to IFN. These analyses revealed that VZV ORF61p substituted for some of ICP0's functions but that it did not complement all of the defects in dl1403 as evidenced by HSV-ORF61's failure to recapitulate the wild-type IFN resistant phenotype.

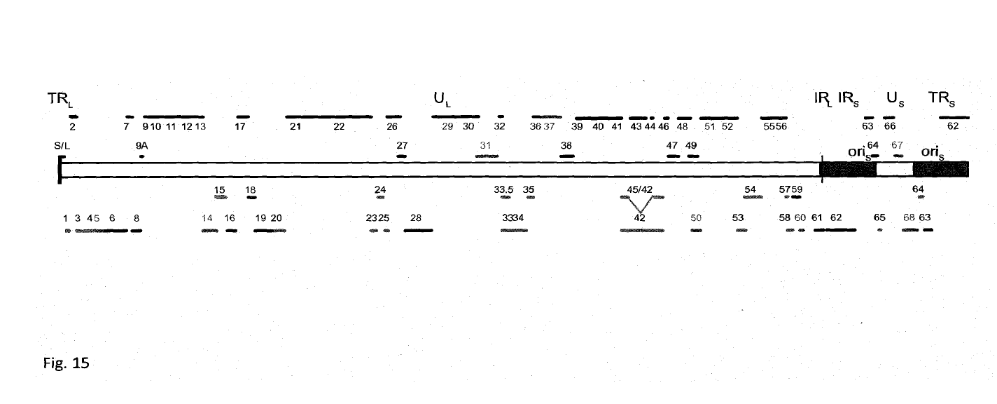

This recombinant virus has characteristics that recapitulate the IFN sensitivity of VZV Oka vaccine strain. VZV encodes 8 glycoproteins that represent the major neutralizing virus epitopes (FIG. 15). The glycoprotein encoding the Us region of HSV can be substituted with the predominant glycoprotein genes ORFs and 68 that are located in the corresponding Us region of VZV. This is done using an HSV-1 bacmid containing ORF61p in place of ICP0 coding sequences. The recombinant construct is transfected into cells to create virus which is then recovered. As known in the art, a recombinant virus can be constructed by introducing simultaneously the desired DNA fragment in a plasmid/bacmid into the host cell along with the virus to permit recombination therewith and recombinant virus production. One by one substitution of HSV glycoproteins with the corresponding sequences from VZV to can be performed to reconstitute an HSV strain whose glycoprotein genes are fully substituted for with VZV glycoprotein genes. This offers improved stability over native VZV and can be grown to higher titers for vaccine production.

### REFERENCES

1. Ambagala, A. P., and J. I. Cohen. 2007. Varicella-Zoster virus IE63, a major viral latency protein, is required to inhibit the alpha interferon-induced antiviral response. J Virol 81:7844-51.
2. Ascoli, C. A., and G. G. Maul. 1991. Identification of a novel nuclear domain. J Cell Biol 112:785-95.
3. Borden, K. L., and P. S. Freemont. 1996. The RING finger domain: a recent example of a sequence-structure family. Curr Opin Struct Biol 6:395-401.
4. Boutell, C., S. Sadis, and R. D. Everett. 2002. Herpes simplex virus type 1 immediate-early protein ICP0 and is isolated RING finger domain act as ubiquitin E3 ligases in vitro. J Virol 76:841-50.
5. Canning, M., C. Boutell, J. Parkinson, and R. D. Everett. 2004. A RING finger ubiquitin ligase is protected from autocatalyzed ubiquitination and degradation by binding to ubiquitin-specific protease USP7. J Biol Chem 279:38160-8.
6. Carvalho, T., J. S. Seeler, K. Ohman, P. Jordan, U. Pettersson, G. Akusjarvi, M. Carmo-Fonseca, and A. Dejean. 1995. Targeting of adenovirus E1A and E4-ORF3 proteins to nuclear matrix-associated PML bodies. J Cell Biol 131:45-56.
7. Chee, A. V., P. Lopez, P. P. Pandolfi, and B. Roizman. 2003. Promyelocytic leukemia protein mediates interferon-based anti-herpes simplex virus 1 effects. J Virol 77:7101-5.
8. Chelbi-Alix, M. K., and H. de Thé. 1999. Herpes virus induced proteasome-dependent degradation of the nuclear bodies-associated PML and Sp100 proteins. Oncogene 18:935-41.
9. Davison, A. J., and J. E. Scott. 1986. The complete DNA sequence of varicella-zoster virus. J Gen Virol 67 (Pt 9):1759-816.
10. Doucas, V., A. M. Ishov, A. Romo, H. Juguilon, M. D. Weitzman, R. M. Evans, and G. G. Maul. 1996. Adenovirus replication is coupled with the dynamic properties of the PML nuclear structure. Genes Dev 10:196-207.
11. Eisfeld, A. J., S. E. Turse, S. A. Jackson, E. C. Lerner, and P. R. Kinchington. 2006. Phosphorylation of the varicella-zoster virus (VZV) major transcriptional regulatory protein IE62 by the VZV open reading frame 66 protein kinase. J Virol 80:1710-23.
12. Everett, R. D. 1985. Activation of cellular promoters during herpes virus infection of biochemically transformed cells. EMBO J 4:1973-80.
13. Everett, R. D. 1989. Construction and characterization of herpes simplex virus type 1 mutants with defined lesions in immediate early gene 1. J Gen Virol 70 (Pt 5):1185-202.
14. Everett, R. D. 2006. Interactions between DNA viruses, ND10 and the DNA damage response. Cell Microbiol 8:365-74.
15. Everett, R. D., W. C. Earnshaw, J. Findlay, and P. Lomonte. 1999. Specific destruction of kinetochore protein CENP-C and disruption of cell division by herpes simplex virus immediate-early protein Vmw110. EMBO J 18:1526-38.
16. Everett, R. D., P. Freemont, H. Saitoh, M. Dasso, A. Orr, M. Kathoria, and J. Parkinson. 1998. The disruption of ND10 during herpes simplex virus infection correlates with the Vmw110- and proteasome-dependent loss of several PML isoforms. J Virol 72:6581-91.
17. Everett, R. D., P. Lomonte, T. Sternsdorf, R. van Driel, and A. Orr. 1999. Cell cycle regulation of PML modification and ND10 composition. J Cell Sci 112 (Pt 24):4581-8.
18. Everett, R. D., and G. G. Maul. 1994. HSV-1 IE protein Vmw110 causes redistribution of PML. Embo J 13:5062-9.
19. Everett, R. D., M. Meredith, and A. Orr. 1999. The ability of herpes simplex virus type 1 immediate-early protein Vmw110 to bind to a ubiquitin-specific protease contributes to its roles in the activation of gene expression and stimulation of virus replication. J Virol 73:417-26.
20. Everett, R. D., M. Meredith, A. Orr, A. Cross, M. Kathoria, and J. Parkinson. 1997. A novel ubiquitin-specific protease is dynamically associated with the PML nuclear domain and binds to a herpesvirus regulatory protein. EMBO J 16:566-77.
21. Everett, R. D., C. Parada, P. Gripon, H. Sirma, and A. Orr. 2008. Replication of ICP0-null mutant herpes simplex virus type 1 is restricted by both PML and Sp100. J Virol 82:2661-72.
22. Everett, R. D., S. Rechter, P. Papior, N. Tavalai, T. Stamminger, and A. Orr. 2006. PML contributes to a cellular mechanism of repression of herpes simplex virus type 1 infection that is inactivated by ICP0. J Virol 80:7995-8005.
23. Gelman, I. H., and S. Silverstein. 1985. Identification of immediate early genes from herpes simplex virus that transactivate the virus thymidine kinase gene. Proc Natl Acad Sci USA 82:5265-9.
24. Grose, C., and P. A. Brunel. 1978. Varicella-zoster virus: isolation and propagation in human melanoma cells at 36 and 32 degrees C. Infect Immun 19:199-203.
25. Grotzinger, T., T. Sternsdorf, K. Jensen, and H. Will. 1996. Interferon-modulated expression of genes encoding the nuclear-dot-associated proteins Sp100 and promyelocytic leukemia protein (PML). Eur J Biochem 238:554-60.
26. Gu, H., and B. Roizman. 2003. The degradation of promyelocytic leukemia and Sp100 proteins by herpes simplex virus 1 is mediated by the ubiquitin-conjugating enzyme UbcH5a. Proc Natl Acad Sci USA 100:8963-8.
27. Guldner, H. H., C. Szostecki, P. Schroder, U. Matschl, K. Jensen, C. Luders, H. Will, and T. Sternsdorf. 1999. Splice variants of the nuclear dot-associated Sp100 protein contain homologies to HMG-1 and a human nuclear phosphoprotein-box motif. J Cell Sci 112 (Pt 5):733-47.
28. Hagglund, R., and B. Roizman. 2002. Characterization of the novel E3 ubiquitin ligase encoded in exon 3 of herpes simplex virus-1-infected cell protein 0. Proc Natl Acad Sci USA 99:7889-94.
29. Hagglund, R., and B. Roizman. 2003. Herpes simplex virus 1 mutant in which the ICP0 HUL-1 E3 ubiquitin ligase site is disrupted stabilizes cdc34 but degrades D-type cyclins and exhibits diminished neurotoxicity. J Virol 77:13194-202.
30. Hagglund, R., and B. Roizman. 2004. Role of ICP0 in the strategy of conquest of the host cell by herpes simplex virus 1. J Virol 78:2169-78.
31. Hagglund, R., C. Van Sant, P. Lopez, and B. Roizman. 2002. Herpes simplex virus 1-infected cell protein 0 contains two E3 ubiquitin ligase sites specific for different E2 ubiquitin-conjugating enzymes. Proc Natl Acad Sci USA 99:631-6.
32. Ishov, A. M., and G. G. Maul. 1996. The periphery of nuclear domain 10 (ND10) as site of DNA virus deposition. J Cell Biol 134:815-26.
33. Ishov, A. M., R. M. Stenberg, and G. G. Maul. 1997. Human cytomegalovirus immediate early interaction with host nuclear structures: definition of an immediate transcript environment. J Cell Biol 138:5-16.
34. Ishov, A. M., O. V. Vladimirova, and G. G. Maul. 2002. Daxx-mediated accumulation of human cytomegalovirus tegument protein pp 71 at ND10 facilitates initiation of viral infection at these nuclear domains. J Virol 76:7705-12.
35. Jul-Larsen, A., T. Visted, B. O. Karlsen, C. H. Rinaldo, R. Bjerkvig, P. E. Lonning, and S. O. Boe. 2004. PML-nuclear bodies accumulate DNA in response to polyomavirus BK and simian virus 40 replication. Exp Cell Res 298:58-73.
36. Koyama, K., and J. Osame. 2000. Stabilized live vaccine. U.S. Pat. No. 6,039,958.
37. Kyratsous, C. A., and S. J. Silverstein. 2007. BAG3, a host cochaperone, facilitates varicella-zoster virus replication. J Virol 81:7491-503.
38. Kyratsous, C. A., and S. J. Silverstein. 2008. The co-chaperone BAG3 regulates Herpes Simplex Virus replication. Proc Natl Acad Sci USA 105:20912-7.
39. Laemmli, U. K. 1970. Cleavage of structural proteins during the assembly of the head of bacteriophage T4. Nature 227:680-5.
40. Lium, E. K., C. A. Panagiotidis, X. Wen, and S. Silverstein. 1996. Repression of the alpha0 gene by ICP4 during a productive herpes simplex virus infection. J Virol 70:3488-96.
41. Lomonte, P., K. F. Sullivan, and R. D. Everett. 2001. Degradation of nucleosome-associated centromeric histone H3-like protein CENP-A induced by herpes simplex virus type 1 protein ICP0. J Biol Chem 276:5829-35.
42. Lorick, K. L., J. P. Jensen, S. Fang, A. M. Ong, S. Hatakeyama, and A. M. Weissman. 1999. RING fingers mediate ubiquitin-conjugating enzyme (E2)-dependent ubiquitination. Proc Natl Acad Sci USA 96:11364-9.
43. Lungu, O., C. A. Panagiotidis, P. W. Annunziato, A. A. Gershon, and S. J. Silverstein. 1998. Aberrant intracellular localization of Varicella-Zoster virus regulatory proteins during latency. Proc Natl Acad Sci USA 95:7080-5.
44. Markowitz, D., S. Goff, and A. Bank. 1988. A safe packaging line for gene transfer: separating viral genes on two different plasmids. J Virol 62:1120-4.
45. Maul, G. G., and R. D. Everett. 1994. The nuclear location of PML, a cellular member of the C3HC4 zinc-binding domain protein family, is rearranged during herpes simplex virus infection by the C3HC4 viral protein ICP0. J Gen Virol 75 (Pt 6):1223-33.
46. Maul, G. G., H. H. Guldner, and J. G. Spivack. 1993. Modification of discrete nuclear domains induced by herpes simplex virus type 1 immediate early gene 1 product (ICP0). J Gen Virol 74 (Pt 12):2679-90.
47. Maul, G. G., A. M. Ishov, and R. D. Everett. 1996. Nuclear domain 10 as preexisting potential replication start sites of herpes simplex virus type-1. Virology 217:67-75.
48. Moriuchi, H., M. Moriuchi, and J. I. Cohen. 1994. The RING finger domain of the varicella-zoster virus open reading frame 61 protein is required for its transregulatory functions. Virology 205:238-46.
49. Moriuchi, H., M. Moriuchi, H. A. Smith, S. E. Straus, and J. I. Cohen. 1992. Varicella-zoster virus open reading frame 61 protein is functionally homologous to herpes simplex virus type 1 ICP0. J Virol 66:7303-8.
50. Moriuchi, H., M. Moriuchi, S. E. Straus, and J. I. Cohen. 1993. Varicella-zoster virus (VZV) open reading frame 61 protein transactivates VZV gene promoters and enhances the infectivity of VZV DNA. J Virol 67:4290-5.
51. Mossman, K. L., H. A. Saffran, and J. R. Smiley. 2000. Herpes simplex virus ICP0 mutants are hypersensitive to interferon. J Virol 74:2052-6.
52. Mullen, M. A., D. M. Ciufo, and G. S. Hayward. 1994. Mapping of intracellular localization domains and evidence for colocalization interactions between the IE110 and IE175 nuclear transactivator proteins of herpes simplex virus. J Virol 68:3250-66.
53. Muller, S., and A. Dejean. 1999. Viral immediate-early proteins abrogate the modification by SUMO-1 of PML and Sp100 proteins, correlating with nuclear body disruption. J Virol 73:5137-43.
54. Nisole, S., J. P. Stoye, and A. Saïb. 2005. TRIM family proteins: retroviral restriction and antiviral defence. Nat Rev Microbiol 3:799-808.
55. Parkinson, J., and R. D. Everett. 2000. Alphaherpesvirus proteins related to herpes simplex virus type 1 ICP0 affect cellular structures and proteins. J Virol 74:10006-17.
56. Pereira, L., M. H. Wolff, M. Fenwick, and B. Roizman. 1977. Regulation of herpesvirus macromolecular synthesis. V. Properties of alpha polypeptides made in HSV-1 and HSV-2 infected cells. Virology 77:733-49.
57. Perry, L. J., F. J. Rixon, R. D. Everett, M. C. Frame, and D. J. McGeoch. 1986. Characterization of the IE110 gene of herpes simplex virus type 1. J Gen Virol 67 (Pt 11):2365-80.
58. Provost, P. J., D. L. Krah, and P. A. Friedman. 1997. Process for attenuated varicella zoster virus vaccine production. U.S. Pat. No. 5,607,852.
59. Quinlan, M. P., and D. M. Knipe. 1985. Stimulation of expression of a herpes simplex virus DNA-binding protein by two viral functions. Mol Cell Biol 5:957-63.
60. Regad, T., and M. K. Chelbi-Alix. 2001. Role and fate of PML nuclear bodies in response to interferon and viral infections. Oncogene 20:7274-86.
61. Sourvinos, G., and R. D. Everett. 2002. Visualization of parental HSV-1 genomes and replication compartments in association with ND10 in live infected cells. EMKO J 21:4989-97.
62. Sternsdorf, T., T. Grotzinger, R. Jensen, and H. Will. 1997. Nuclear dots: actors on many stages. Immunobiology 198:307-31.
63. Stevenson, D., K. L. Colman, and A. J. Davison. 1994. Delineation of a sequence required for nuclear localization of the protein encoded by varicella-zoster virus gene 61. J Gen Virol 75 (Pt 11):3229-33.
64. Stow, N. D., and E. C. Stow. 1986. Isolation and characterization of a herpes simplex virus type 1 mutant containing a deletion within the gene encoding the immediate early polypeptide Vmw110. J Gen Virol 67 (Pt 12):2571-85.
65. Stuurman, N., A. de Graaf, A. Floore, A. Josso, B. Humbel, L. de Jong, and R. van Driel. 1992. A monoclonal antibody recognizing nuclear matrix-associated nuclear bodies. J Cell Sci 101 (Pt 4):773-84.
66. Szostecki, C., H. H. Guldner, H. J. Netter, and H. Will. 1990. Isolation and characterization of cDNA encoding a human nuclear antigen predominantly recognized by autoantibodies from patients with primary biliary cirrhosis. J Immunol 145:4338-47.
67. Tavalai, N., P. Papior, S. Rechter, M. Leis, and T. Stamminger. 2006. Evidence for a role of the cellular ND10 protein PML in mediating intrinsic immunity against human cytomegalovirus infections. J Virol 80:8006-18.
68. Tavalai, N., P. Papior, S. Rechter, and T. Stamminger. 2008. Nuclear domain 10 components promyelocytic leukemia protein and hDaxx independently contribute to an intrinsic antiviral defense against human cytomegalovirus infection. J Virol 82:126-37.
69. Tavalai, N., P. Papior, S. Rechter, and T. Stamminger. 2007. The ND10 components PML and hDaxx independently contribute to an intrinsic antiviral defense against human cytomegalovirus infection. J. Virol.
70. Tavalai, N., and T. Stamminger. 2008. New insights into the role of the subnuclear structure ND10 for viral infection. Biochim Biophys Acta 1783:2207-21.
71. Tzanck, A. 1947. Le cytodiagnostic immediat en dermatologie. Bull Soc Fr Dermatol Syph 7:68.
72. Ullman, A. J., and P. Hearing. 2008. Cellular proteins PML and Daxx mediate an innate antiviral defense antagonized by the adenovirus E4 ORF3 protein. J Virol 82:7325-35.
73. Ullman, A. J., N. C. Reich, and P. Hearing. 2007. Adenovirus E4 ORF3 protein inhibits the interferon-mediated antiviral response. J Virol 81:4744-52.
74. Walters, M. S., C. A. Kyratsous, S. Wan, and S. Silverstein. 2008. Nuclear import of the varicella-zoster virus latency-associated protein ORF63 in primary neurons requires expression of the lytic protein ORF61 and occurs in a proteasome-dependent manner. J Virol 82:8673-86.
75. Yee, J. K., A. Miyanohara, P. LaPorte, K. Bouic, J. C. Burns, and T. Friedmann. 1994. A general method for the generation of high-titer, pantropic retroviral vectors: highly efficient infection of primary hepatocytes. Proc Natl Acad Sci USA 91:9564-8.
76. Zhu, X. X., C. S. Young, and S. Silverstein. 1988. Adenovirus vector expressing functional herpes simplex virus ICP0. J Virol 62:4544-53.
77. Ambagala, A. P., and J. I. Cohen. 2007. Varicella-Zoster virus IE63, a major viral latency protein, is required to inhibit the alpha interferon-induced antiviral response. J Virol 81:7844-51.
78. Cai, W., and P. A. Schaffer. 1992. Herpes simplex virus type 1 ICP0 regulates expression of immediate-early, early, and late genes in productively infected cells. J Virol 66:2904-15.
79. Chee, A. V., P. Lopez, P. P. Pandolfi, and B. Roizman. 2003. Promyelocytic leukemia protein mediates interferon-based anti-herpes simplex virus 1 effects. J Virol 77:7101-5.
80. Chen, J., and S. Silverstein. 1992. Herpes simplex viruses with mutations in the gene encoding ICP0 are defective in gene expression. J Virol 66:2916-27.
81. Cliffe, A. R., and D. M. Knipe. 2008. Herpes simplex virus ICP0 promotes both histone removal and acetylation on viral DNA during lytic infection. J Virol 82:12030-8.
82. Emeny, J. M., and M. J. Morgan. 1979. Regulation of the interferon system: evidence that Vero cells have a genetic defect in interferon production. J Gen Virol 43:247-52.
83. Everett, R. D. 1985. Activation of cellular promoters during herpes virus infection of biochemically transformed cells. EMBO J 4:1973-80.
84. Everett, R. D. 1989. Construction and characterization of herpes simplex virus type 1 mutants with defined lesions in immediate early gene 1. J Gen Virol 70 (Pt 5):1185-202.
85. Everett, R. D., C. Parada, P. Gripon, H. Sirma, and A. Orr. 2008. Replication of ICP0-null mutant herpes simplex virus type 1 is restricted by both PML and Sp100. J Virol 82:2661-72.
86. Everett, R. D., S. Rechter, P. Papior, N. Tavalai, T. Stamminger, and A. Orr. 2006. PML contributes to a cellular mechanism of repression of herpes simplex virus type 1 infection that is inactivated by ICP0. J Virol 80:7995-8005.
87. Gelman, I. H., and S. Silverstein. 1985. Identification of immediate early genes from herpes simplex virus that transactivate the virus thymidine kinase gene. Proc Natl Acad Sci USA 82:5265-9.
88. Gu, H., and B. Roizman. 2007. Herpes simplex virus-infected cell protein 0 blocks the silencing of viral DNA by dissociating histone deacetylases from the CoREST-REST complex. Proc Natl Acad Sci USA 104:17134-9.
89. Guldner, H. H., C. Szostecki, P. Schroder, U. Matschl, K. Jensen, C. Luders, H. Will, and T. Sternsdorf. 1999. Splice variants of the nuclear dot-associated Sp100 protein contain homologies to HMG-1 and a human nuclear phosphoprotein-box motif. J Cell Sci 112 (Pt 5):733-47.
90. Hirt, B. 1967. Selective extraction of polyoma DNA from infected mouse cell cultures. J Mol Biol 26:365-9.
91. Kyratsous, C. A., and S. J. Silverstein. 2007. BAG3, a host cochaperone, facilitates varicella-zoster virus replication. J Virol 81:7491-503.
92. Kyratsous, C. A., and S. J. Silverstein. 2009. Components of nuclear domain 10 bodies regulate varicella-zoster virus replication. J Virol 83:4262-74.
93. Kyratsous, C. A., and S. J. Silverstein. 2008. The co-chaperone BAG3 regulates Herpes Simplex Virus replication. Proc Natl Acad Sci USA 105:20912-7.
94. Laemmli, U. K. 1970. Cleavage of structural proteins during the assembly of the head of bacteriophage T4. Nature 227:680-5.
95. Liu, X., E. Broberg, D. Watanabe, T. Dudek, N. Deluca, and D. M. Knipe. 2009. Genetic engineering of a modified herpes simplex virus 1 vaccine vector. Vaccine 27:2760-7.
96. Lium, E. K., C. A. Panagiotidis, X. Wen, and S. Silverstein. 1996. Repression of the alpha0 gene by ICP4 during a productive herpes simplex virus infection. J Virol 70:3488-96.
97. Lium, E. K., and S. Silverstein. 1997. Mutational analysis of the herpes simplex virus type 1 ICP0 C3HC4 zinc ring finger reveals a requirement for ICP0 in the expression of the essential alpha27 gene. J Virol 71:8602-14.
98. Maul, G. G. 1998. Nuclear domain 10, the site of DNA virus transcription and replication. Bioessays 20:660-7.
99. Maul, G. G., H. H. Guldner, and J. G. Spivack. 1993. Modification of discrete nuclear domains induced by herpes simplex virus type 1 immediate early gene 1 product (ICP0). J Gen Virol 74 (Pt 12):2679-90.
100. Moriuchi, H., M. Moriuchi, H. A. Smith, S. E. Straus, and J. I. Cohen. 1992. Varicella-zoster virus open reading frame 61 protein is functionally homologous to herpes simplex virus type 1 ICP0. J Virol 66:7303-8.
101. Moriuchi, H., M. Moriuchi, S. E. Straus, and J. I. Cohen. 1993. Varicella-zoster virus (VZV) open reading frame 61 protein transactivates VZV gene promoters and enhances the infectivity of VZV DNA. J Virol 67:4290-5.
102. Mossman, K. L., H. A. Saffran, and J. R. Smiley. 2000. Herpes simplex virus ICP0 mutants are hypersensitive to interferon. J Virol 74:2052-6.
103. Parkinson, J., and R. D. Everett. 2000. Alphaherpesvirus proteins related to herpes simplex virus type 1 ICP0 affect cellular structures and proteins. J Virol 74:10006-17.
104. Pereira, L., M. H. Wolff, M. Fenwick, and B. Roizman. 1977. Regulation of herpesvirus macromolecular synthesis. V. Properties of alpha polypeptides made in HSV-1 and HSV-2 infected cells. Virology 77:733-49.
105. Quinlan, M. P., and D. M. Knipe. 1985. Stimulation of expression of a herpes simplex virus DNA-binding protein by two viral functions. Mol Cell Biol 5:957-63.
106. Samaniego, L. A., N. Wu, and N. A. DeLuca. 1997. The herpes simplex virus immediate-early protein ICP0 affects transcription from the viral genome and infected-cell survival in the absence of ICP4 and ICP27. J Virol 71:4614-25.
107. Smiley, J. R. 1980. Construction in vitro and rescue of a thymidine kinase-deficient deletion mutation of herpes simplex virus. Nature 285:333-5.
108. Sternsdorf, T., K. Jensen, and H. Will. 1997. Evidence for covalent modification of the nuclear dot-associated proteins PML and Sp100 by PIC1/SUMO-1. J Cell Biol 139:1621-34.
109. Stow, N. D., and E. C. Stow. 1986. Isolation and characterization of a herpes simplex virus type 1 mutant containing a deletion within the gene encoding the immediate early polypeptide Vmw110. J Gen Virol 67 (Pt 12):2571-85.
110. Tavalai, N., P. Papior, S. Rechter, M. Leis, and T. Stamminger. 2006. Evidence for a role of the cellular ND10 protein PML in mediating intrinsic immunity against human cytomegalovirus infections. J Virol 80:8006-18.
111. Walters, M. S., C. A. Kyratsous, S. Wan, and S. Silverstein. 2008. Nuclear import of the varicella-zoster virus latency-associated protein ORF63 in primary neurons requires expression of the lytic protein ORF61 and occurs in a proteasome-dependent manner. J Virol 82:8673-86.
112. Yao, F., and P. A. Schaffer. 1995. An activity specified by the osteosarcoma line U2OS can substitute functionally for ICP0, a major regulatory protein of herpes simplex virus type 1. J Virol 69:6249-58.
113. Zhu, X. X., J. X. Chen, and S. Silverstein. 1991. Isolation and characterization of a functional cDNA encoding ICP0 from herpes simplex virus type 1. J Virol 65:957-60.

1. A recombinant deoxyribonucleic acid comprising a human herpes simplex virus (HSV) deoxyribonucleic acid (DNA) having a heterologous DNA integrated therein wherein the heterologous DNA encodes a polypeptide comprising a RING-finger domain.

2. The recombinant deoxyribonucleic acid of claim 1, wherein the HSV DNA is genomic DNA and the heterologous DNA is integrated into the HSV DNA in place of a portion of genomic HSV DNA which encodes a HSV Infected Cell Polypeptide 0 (ICP0).

3. The recombinant deoxyribonucleic acid of claim 2, wherein the heterologous DNA is inserted into the genomic HSV DNA between a HSV ICPO 5′ untranslated region (UTR) and a HSV ICPO 3′ untranslated region (UTR).

4. The recombinant deoxyribonucleic acid of claim 1, wherein the polypeptide comprising the RING-finger domain has the amino acid sequence of varicella zoster virus ORF61 protein.

5. The recombinant deoxyribonucleic acid of claim 1, wherein the heterologous polypeptide comprising the RING-finger domain has the amino acid sequence of a equine herpes virus, bovine herpes virus, or pseudorabies virus protein.

6. The recombinant deoxyribonucleic acid of claim 4, wherein the HSV DNA is a HSV genome.

7. The recombinant deoxyribonucleic acid of claim 1 further comprising a heterologous DNA encoding a glycoprotein.

8. The recombinant deoxyribonucleic acid of claim 7, wherein the glycoprotein has the amino acid sequence of a varicella zoster virus glycoprotein.

9. The recombinant deoxyribonucleic acid of claim 8, wherein the glycoprotein comprises a varicella zoster virus neutralizing epitope.

10. The recombinant deoxyribonucleic acid of claim 1, wherein no polypeptide encoded by the recombinant deoxyribonucleic acid degrades mammalian promyelocytic leukemia protein (PML).

11. The recombinant deoxyribonucleic acid of claim 1, wherein the heterologous DNA encoding the polypeptide is integrated such that the polypeptide is expressed when the recombinant deoxyribonucleic acid is integrated into a genome of a suitable host cell.

12. The recombinant deoxyribonucleic acid of claim 7, wherein the heterologous DNA encoding the glycoprotein is inserted into a non-essential gene or region of the HSV DNA.

13. A recombinant human herpes simplex virus (HSV) comprising a heterologous DNA encoding a polypeptide comprising a RING-finger domain which heterologous DNA is (a) inserted into a non-essential region of the HSV genome, and (b) expressed in a host cell into which the recombinant HSV is introduced.

14. The recombinant HSV of claim 13, wherein the heterologous DNA is integrated into the HSV genome in place of a portion of the HSV genome which encodes a HSV Infected Cell Polypeptide 0 (ICP0).

15. The recombinant HSV of claim 13, wherein the heterologous DNA is inserted into the HSV genome between a HSV ICPO 5′ untranslated region (UTR) and a HSV ICPO 3′ untranslated region (UTR).

16. The recombinant HSV of claim 13, wherein the polypeptide comprising the RING-finger domain has the amino acid sequence of varicella zoster virus ORF61 protein.

17. The recombinant HSV of claim 13, wherein the polypeptide comprising the RING-finger domain has the amino acid sequence of a equine herpes virus, bovine herpes virus, or pseudorabies virus protein.

18. The recombinant HSV of claim 13 further comprising a heterologous DNA encoding a glycoprotein and inserted into a non-essential region of the HSV genome.

19. The recombinant HSV of claim 18, wherein the glycoprotein has the amino acid sequence of a varicella zoster virus glycoprotein.

20. The recombinant HSV of claim 18, wherein the glycoprotein comprises a varicella-zoster virus neutralizing epitope.

21. The recombinant HSV of claim 18, wherein the heterologous DNA encoding the glycoprotein is inserted into a non-essential gene or region of the HSV genome.

22. The recombinant HSV of claim 13, wherein none of the polypeptides encoded by the recombinant HSV genome degrade PML.

23. The recombinant HSV of claim 13, comprising up to eight different heterologous DNAs, each encoding a different varicella zoster virus glycoprotein.

24. A vaccine comprising (1) a pharmaceutically acceptable carrier and (2) a recombinant virus which comprises a recombinant deoxyribonucleic acid comprising a human herpes simplex virus (HSV) genome having (a) a heterologous DNA encoding a polypeptide comprising a RING-finger domain integrated therein and (b) having one or more heterologous DNAs, each encoding a glycoprotein, integrated therein.

25. The vaccine of claim 24, wherein the heterologous DNA is integrated into the HSV genome in place of a portion of HSV DNA encoding a HSV Infected Cell Polypeptide 0 (ICP0).

26. The vaccine of claim 24, wherein the heterologous DNA is inserted into the HSV genome between a HSV ICPO 5′ untranslated region (UTR) and a HSV ICPO 3′ untranslated region (UTR).

27. The vaccine of claim 24, wherein the heterologous polypeptide comprising a RING-finger domain has the amino acid sequence of varicella zoster virus ORF61 protein.

28. The vaccine of claim 24, wherein the heterologous polypeptide comprising a RING-finger domain has the amino acid sequence of EHV, BHV, or pseudorabies virus protein.

29. The vaccine of claim 24, wherein the glycoprotein has the amino acid sequence of a varicella zoster virus glycoprotein.

30. The vaccine of claim 24, wherein the glycoprotein comprises a varicella-zoster virus neutralizing epitope.

31. The vaccine of claim 24, wherein the heterologous DNAs encoding the glycoproteins and the a heterologous DNA encoding the polypeptide comprising the RING-finger domain are each inserted into non-essential genes or regions of the HSV genome.

32. A method of immunizing a subject against a varicella zoster virus infection comprising administering to the subject an amount of the vaccine of claim 26 effective to elicit a immune response the varicella zoster virus in the subject and thereby effect immunization of the subject.

33. A method for preparing a composition useful for preventing infection by a virus, comprising combining a recombinant virus of claim 13 with a live vaccine stabilizer, so as to prepare the composition.


SEQUENCE LISTING (8 sequences)

SEQ ID NO: 1 - protein, 775 aa, human herpesvirus
MEPRPGASTRRPEGRPQREPAPDVWVFPCDRDLPDSSDSEAETEVGGRGDADHHDDDSASEADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAAPRGASGPSGGVGVGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPLPSAASVEQEAAVRPRKRRGSGQENPSPQSTRPPLAPAGAKRAATHPPSDSGPGGRGQGGPGTPLTSSAASASSSSASSSSAPTPAGAASSAAGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVMPPEYPTAPASEWNSLWMTPVGNMLFDQGTLVGALDFRSLRSRHPWSGEQGASTRDEGKQ

SEQ ID NO: 2 - protein, 467 aa, human herpesvirus
MDTILAGGSGTSDASDNTCTICMSTVSDLGKTMPCLHDFCFVCIRAWTSTSVQCPLCRCPVQSILHKIVSDTSYKEYEVHPSDDDGFSEPSFEDSIDILPGDVIDLLPPSPGPSRESIQQPTSRSSREPIQSPNPGPLQSSAREPTAESPSDSQQDSIQPPTRDSSPGVTKTCSTASFLRKVFFKDQPAVRSATPVVYGSIESAQQPRTGGQDYRDRPVSVGINQDPRTMDRLPFRATDRGTEGNARFPCYMQPLLGWLDDQLAELYQPEIVEPTKMLILNYIGIYGRDEAGLKTSLRCLLHDSTGPFVTNMLFLLDRCTDPTRLTMQTWTWKDTAIQLITGPIVRPETTSTGETSRGDERDTRLVNTPQKVRLFSVLPGIKPGSARGAKRRLFHTGRDVKRCLTIDLTSESDSACKGSKTRKVASPQGESNTPSTSGSTSGSLKHLTKKSSAGKAGKGIPNKMKKS

SEQ ID NO: 3 - DNA, 20 bp, artificial sequence
  primer directed to human herpesvirus ORF61
acagaagccccgcctacgtt

SEQ ID NO: 4 - DNA, 23 bp, artificial sequence
  primer directed to human herpesvirus ORF61
ggtgcccgtgtctttcacttttc

SEQ ID NO: 5 - DNA, 32 bp, artificial sequence
  primer directed to human herpesvirus ORF61
gggaattcggggccccttcaatcgtcggctag

SEQ ID NO: 6 - DNA, 33 bp, artificial sequence
  primer directed to human herpesvirus ORF61
tgcggccgcgaatctcgcgtttccctctgttcc

SEQ ID NO: 7 - DNA, 31 bp, artificial sequence
  primer directed to human herpesvirus ORF61
gggtcgacttgcattaccctatcccagtatt

SEQ ID NO: 8 - DNA, 29 bp, artificial sequence
  primer directed to human herpesvirus ORF61
ccgtcgaccccaacaaactaggacttctt